(19) United States
(12) Patent Application Publication
(10) Pub. No.: US 2020/0046287 A1
(43) Pub. Date: Feb. 13, 2020

(54) METHOD AND SYSTEM FOR CARDIOVASCULAR RISK ASSESSMENT USING PROGRAMMED ACTIVITY OF DAILY LIVING

(71) Applicant: UTI Limited Partnership, Calgary (CA)

(72) Inventors: Henry J. DUFF, Calgary (CA); Daniel E. ROACH, Calgary (CA)

(21) Appl. No.: 16/056,921

(22) Filed: Aug. 7, 2018

(51) Int. Cl. A61B 5/00; A61B 5/02; A61B 5/021; A61B 5/024

(52) U.S. Cl. CPC A61B 5/4884; A61B 5/02028; A61B 5/021; A61B 2560/0475; A61B 5/742; A61B 5/0002; A61B 5/02405

(57) ABSTRACT

Medical methods and systems relating to cardiovascular risk and/or performance assessment are provided. An individual performs a programmed activity of daily living such as reading displayed information aloud. The reading aloud is a temporary micro physiological challenge to the individual. The intensity of the micro challenges is gently increased over a short session. The cardiovascular (CV) response of the individual is measured during the session and is analyzed to assess a CV risk or performance of the individual. The existence of subtle CV deviations identified in the CV response of the individual may indicate a CV risk.

### FIELD

The present disclosure relates to medical methods and devices, and more particularly to methods and systems relating to cardiovascular risk and/or performance assessment.

### BACKGROUND

The market for home-based portable systems that acquire, analyze and compile physiological data is flourishing. This growth marks the transition from historic hospital-based medical testing/risk assessment to home-based, self-testing and longitudinal performance/risk monitoring. This transition is not simply about miniaturizing the classic physiological sensors for home use, it is also about using new multi-channel data acquisition techniques designed in concert with new signal processing technologies to optimize extraction of very specific physiological information. Classic one-day recordings of electrocardiograms (ECG) using systems such as a Holter monitor, or the 24-hour recordings of spot average blood pressure (BP) will still be useful for screening patients as in the past. These classic approaches to cardiovascular (CV) risk assessment have deficiencies including: unknown deviation pathways to CV events; paucity of longitudinal tools to obtain subtle precursors to CV clinical events; and science has ignored naturally-occurring macro health challenges which often preview future deviations leading to CV events.

Improvements relating to CV risk and/or performance assessment are desired. Improvements in computer-based systems, devices, and methods related to CV risk and/or performance assessment are also desired.

The above information is presented as background information only to assist with an understanding of the present disclosure. No assertion or admission is made as to whether any of the above might be applicable as prior art with regard to the present disclosure.

### SUMMARY

According to an aspect, the present disclosure is directed to a method of assessing health performance of a human, the method comprising displaying sets of information sequentially in time to be read aloud by the human, wherein each set of information is configured to serve as a physiological micro challenge to the human when read aloud, and wherein on average the size of the information in each set of information increases over time thereby increasing the intensity of the micro challenges, measuring a sequence of cardiovascular (CV) responses of the human to each of the physiological micro challenges during a session, each of the CV responses associated in time with the reading aloud of one of the sets of information, comparing at least one of the CV responses to predefined reference CV response information, and determining, based on the comparing, if at least one of the CV responses represents a CV response deviation relative to the reference CV response information.

In an embodiment, the determining involves determining if one or more of the measured CV responses indicates a CV response deviation in the form of a heart beat scale CV abnormality that emerged during the session of measured CV responses, wherein the CV abnormality is abnormal relative to a reference heart beat scale CV response in the reference CV response information.

In an embodiment, the heart beat scale CV abnormality is at least one of a blood pressure alternans or premature ventricular contractions (PVC).

In an embodiment, the measuring a CV response to a given physiological micro challenge involves measuring the CV response spanning a read aloud interval, where a read aloud interval is the time period during which the set of information corresponding to the given micro challenge is read aloud, and the determining involves determining if there is a CV response deviation in the form of an abnormality in the way in which the read aloud interval CV responses of the human change during the session of measured CV responses, where the change is abnormal relative to reference read aloud interval CV response change information.

In an embodiment, the abnormality in the way in which the read aloud interval CV responses of the human change is in the form of at least one of a blood pressure (BP) change and a heart rate (HR) change.

In an embodiment, the determining involves determining if there is a CV response deviation in the form of an average incremental or decremental change in a CV characteristic during the session of measured CV responses that is abnormal relative to reference change information for the CV characteristic.

In an embodiment, the average incremental or decremental change in a CV characteristic includes at least one of a progressive relative tachycardia and a blood pressure progressive hypertension.

In an embodiment, the method further comprises measuring CV responses of the human during breathe-in intervals that interspace read aloud intervals during which the sets of information are read aloud.

In an embodiment, the session terminates upon a first occurrence of a failure of one of the displayed sets of information to be read aloud.

In an embodiment, the termination occurs due to self-termination by the human in response to the intensity of the micro challenges reaching an intolerable level to the human.

In an embodiment, the measuring a sequence of cardiovascular (CV) responses involves measuring a blood pressure of the human.

In an embodiment, the method further comprises generating an output indicating the result of the determination.

According to an aspect, the present disclosure is directed to a system comprising a computer processor, a computer memory in communication with the processor, a display in communication with the processor, a physiological sensor in communication with the processor, wherein the processor, display and sensor are configured to display sets of information sequentially in time to be read aloud by the human, wherein each set of information is configured to serve as a physiological micro challenge to the human when read aloud, and wherein on average the size of the information in each set of information increases over time thereby increasing the intensity of the micro challenges, measure a sequence of cardiovascular (CV) responses of the human to each of the physiological micro challenges during a session, each of the CV responses associated in time with the reading aloud of one of the sets of information, compare at least one of the CV responses to predefined reference CV response information, and determine, based on the comparing, if at least one of the CV responses represent a CV response deviation relative to the reference CV response information.

In an embodiment, the determining involves determining if one or more of the measured CV responses indicates a CV response deviation in the form of a heart beat scale CV abnormality that emerged during the session of measured CV responses, wherein the CV abnormality is abnormal relative to a reference heart beat scale CV response in the reference CV response information.

In an embodiment, the heart beat scale CV abnormality is at least one of a blood pressure alternans or premature ventricular contractions (PVC).

In an embodiment, the measuring a CV response to a given physiological micro challenge involves measuring the CV response spanning a read aloud interval, where a read aloud interval is the time period during which the set of information corresponding to the given micro challenge is read aloud, and the determining involves determining if there is a CV response deviation in the form of an abnormality in the way in which the read aloud interval CV responses of the human change during the session of measured CV responses, where the change is abnormal relative to reference read aloud interval CV response change information.

In an embodiment, the determining involves determining if there is a CV response deviation in the form of an average incremental or decremental change in a CV characteristic during the session of measured CV responses that is abnormal relative to reference change information for the CV characteristic.

In an embodiment, the average incremental or decremental change in a CV characteristic includes at least one of a progressive relative tachycardia and a blood pressure progressive hypertension.

In an embodiment, the system is further configured to measure CV responses of the human during breathe-in intervals that interspace read aloud intervals during which the sets of information are read aloud.

According to an aspect, the present disclosure is directed to a non-transitory computer-readable medium comprising computer readable instructions which, when executed by one or more processors, cause the performance of displaying sets of information sequentially in time to be read aloud by the human, wherein each set of information is configured to serve as a physiological micro challenge to the human when read aloud, and wherein on average the size of the information in each set of information increases over time thereby increasing the intensity of the micro challenges, measuring a sequence of cardiovascular (CV) responses of the human to each of the physiological micro challenges during a session, each of the CV responses associated in time with the reading aloud of one of the sets of information, comparing at least one of the CV responses to predefined reference CV response information, and determining, based on the comparing, if at least one of the CV responses represents a CV response deviation relative to the reference CV response information.

The foregoing summary provides some aspects and features according to the present disclosure but is not intended to be limiting. Other aspects and features of the present disclosure will become apparent to those ordinarily skilled in the art upon review of the following description of specific embodiments in conjunction with the accompanying figures. Accordingly, the drawings and detailed description are to be regarded as illustrative in nature and not restrictive.

### BRIEF DESCRIPTION OF THE DRAWINGS

Embodiments of the present disclosure will now be described, by way of example only, with reference to the attached Figures.

FIG. 1 is a system for assessing cardiovascular risk of an individual using a programmed activity of daily living.

FIG. 2 is a representation of sets of information being displayed sequentially, in time, to be read aloud.

FIGS. 2A-2G are screenshots of a display showing information displayed sequentially in time to be read aloud in a read aloud PADL session according to another embodiment.

FIG. 3 is a process flow diagram of an example PADL according to the present disclosure.

FIG. 4 is a set of graphs showing designated healthy CV response sequences to a read aloud PADL according to the present disclosure.

FIG. 5 is a process flow chart of an example method of a read aloud PADL.

FIG. 6 is a set of graphs showing CV response sequences to a read aloud PADL that reveal a deviation in the form of a SBP alternans.

FIG. 7 is a set of graphs showing CV response sequences to a read aloud PADL that reveal a deviation in the form of premature ventricular contractions (PVCs).

FIG. 8 is a set of graphs showing CV response sequences to a read aloud PADL that reveal a deviation in the form of a lack of bradycardic capacity.

FIG. 9 is a set of graphs showing CV response sequences to a read aloud PADL that reveal a deviation in the form of a sharp inspiratory SBP drop.

FIG. 10 is a set of graphs showing CV response sequences to a read aloud PADL that reveal a deviation in the form of a failure in one or more of diastolic blood pressure (DBP), pulse pressure (PP), and systolic blood pressure (SBP) to modulate in response to the read aloud PADL session.

FIG. 11 is a set of graphs showing CV response sequences to a read aloud PADL that reveal a deviation in the form of progressive hypertension in DBP, PP or SBP.

FIG. 12 is a pair of graphs providing a comparison of delineated alternan subsequences using minimum runs of 2 beats versus 3 beats.

FIG. 13 is a set of graphs showing CV response sequences to a read aloud PADL of an individual with atrial fibrillation/atrial flutter (AF) that reveal a deviation in the form of SBP alternans.

FIG. 14 is a set of graphs showing CV response sequences to a read aloud PADL of an AF individual that reveal a deviation in the form of PVCs.

FIG. 15 is a set of graphs showing CV response sequences to a read aloud PADL of an AF individual that reveal a deviation in the form of a sharp inspiratory SBP drop.

FIG. 16 is a set of graphs showing CV response sequences to a read aloud PADL of an AF individual that reveal a deviation in the form of a progressive hypertension in SBP.

FIG. 17 is a process flow chart of an example method of a read aloud PADL according to the present disclosure.

FIG. 18 is a block diagram of an example system.

FIG. 19 is a block diagram of an example electronic device.

### DETAILED DESCRIPTION

The present disclosure is generally directed to methods and systems related to assessing cardiovascular risk and/or performance. The present disclosure is further directed to improvements in the technological fields of computer based methods, systems, and devices related to assessing cardiovascular risk and/or performance.

With the increasing prevalence of artificial intelligence (AI), there will be an enormous demand for new hybrid physiological metrics that can be easily and repeatedly obtained at home, for all ages and physical capacities, using minimal equipment and requiring no more effort and risk than any normal activity of daily living. However, to extract useful information from data often requires careful and innovative design.

The present disclosure is related to a method that employs one or more programmed activities of daily living to generate and collect data that may be used to assess CV risk or performance of an individual. The individual may be a patient, a test subject, etc. but the general term “individual” is used herein. In an embodiment, the method programmatically increases a temporary micro physiological challenge level to the individual in a gentle manner using a simple activity of daily living (e.g., speaking a phrase out loud). A micro physiological challenge is sometimes referred to as a micro stress. The CV system of the individual typically responds to these small progressive increases in challenge levels. CV data is obtained from the individual during the session of the activity of daily living, which may be analyzed to assess a CV risk or performance of the individual. In an embodiment, a session of PADL begins with the micro challenge being at a relatively low level so that the individual senses or exhibits little or no discernable response to the micro challenge. The intensity of the micro challenges is increased over time during the session thereby potentially revealing any non-normal CV response deviations exhibited by the individual. The session of programmed activity of daily living may end when the individual self-terminates the session, often in response to the micro challenge level reaching an intensity that is no longer tolerable to the individual from a comfort perspective. In an embodiment, the method or system is designed specifically to reveal one or more existing deviations, which may include subtle deviations, from a reference CV response. The reference CV response may include CV response data from a set of designated healthy individuals and in this regard may be considered a normal CV response.

A deviation in a CV response generally refers to a CV response of an individual that differs from a reference CV response such as a normal or healthy CV response. A CV response may deviate from a reference response in one or more different ways and in terms of one or more CV parameters. Furthermore, deviations may occur in isolation or they may be concurrent.

In an embodiment, the activity of daily living involves the reading aloud of information displayed to the individual. Sets of information are sequentially displayed in time to be read aloud, where each set of information serves as a physiological micro challenge or micro stress to the individual when read aloud. On average, the size of the information in each set of information increases over time thereby increasing the intensity of the micro challenges.

A potential advantage of using activities of daily living as a test platform is that the testing itself may become a routine daily activity. The science and mathematics underlying a design of the present read aloud programmed activities of daily living (PADL) was developed in part based on a research study using a set of designated healthy individuals, and individuals with heart failure or those at risk for heart failure. The group of designated healthy individuals and patients totaled 350 persons.

In an embodiment, PADL is a platform for one or more of home-based, self-administered, longitudinal assessment of CV performance/risk profiles.

Furthermore, in embodiments according to the present disclosure described herein, a PADL involves the individual reading aloud of displayed sets of information. It is to be appreciated, however, that the PADL may take other forms and is thus not limited to read aloud PADLs. For example, in other embodiments, the PADL may be in the form of a progressive set of read aloud simple mathematical computations to be made by the individual and then verbalized out loud. In an embodiment, the set of mathematical computations are shown as a set of visual mathematic expressions (e.g., add, subtract) applied during the expiratory phase of breathing. The complexity and/or length of the serial computations are increased progressively thereby potentially revealing the impact of mental micro challenges on CV responses. In another embodiment, the PADL is a walk, for example a 6-minute walk. In an embodiment, the tempo, cadence and speed of the walk is programmed to gradually increase but is normalized to the person's own level of activity of daily living. However, this PADL is distinctly different from a treadmill test in that it is not a submaximal stress test; rather it is an assessment of the physiologic response to a normal activity of daily living. In a different field of activity of daily living, the individual is shown a series of visual pictures of trees going through normal seasonal changes from spring, summer, autumn and winter; the physiological responses to these visual stimuli are recorded. In a more complete embodiment, patients elect to undergo a programmed day, wherein the physiologic responses to programmed upright posture and walking is compared to programmed inactivity. Also, in some embodiments, the PADL may consist of multiple simultaneous PADLs. Also, in some embodiments, the PADL may consist of multiple PADLs.

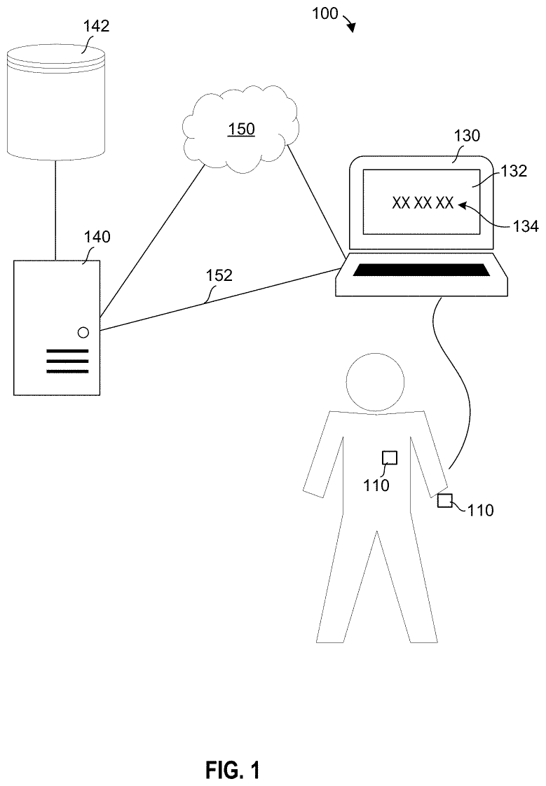

FIG. 1 shows an example system 100 according to an embodiment for collecting CV data from an individual during a PADL session. System 100 may also be used for assessing CV risk and/or CV performance of the individual. System 100 generally comprises a computing device, such as computer 130, configured to cause information 134 to be displayed to the individual in a read aloud PADL session, for example on display 132 of computer 130. Information 134 may comprise sets of information to be displayed sequentially in time to be read aloud by the individual. Further, system 100 generally comprises one or more sensors 110 for obtaining CV measurements or other data from the individual during the PADL session. CV responses of the individual measured during a PADL session are referred to as a sequence of CV responses. The one or more sensors 110 may include a light based sensor (e.g., a 940 nm infra-red LED and photo-transistor pair) that may be placed on the finger of the individual, or any other suitable types of sensors, including but not limited to a capacitance sensor, RF impedance plethysmography, air-cuff plethysmography, accelerometer, electronic micro-strain gauge, ECG, O2-sensors and ultrasonics. One or more sensors 110 may be in communication with computer 130, and computer 130 may receive a signal comprising CV related sensor data from the one or more sensors 110. The data may be in the form of unprocessed analog or digital measurement signals, and/or in the form of signals that have undergone some processing or conversion by sensor 110 prior to transmission to computer 130. The sensor data may be temporarily or permanently stored in a memory of computer 130. Additionally or alternatively, information relating to the CV data or risk assessment may be communicated to another computing device, for example a server 140 possibly paired with a database 142. The communication between computer 130 and server 140 may be over a direct communication link 152 or over a communication network 150.

Computer 130 may be any suitable computing device or set of computing devices, including but not limited to desktop computers, laptop computers, tablet computers, smart phones, or smart watches.

One or more sensors 110 may be of any suitable type or types, including of types for obtaining CV measurements of an individual. A sensor 110 may be adapted to measure blood pressure of the individual, for example by sensing raw pulsatile waveforms. Key markers may then be determined from the raw blood pressure data, such as one or more of R-R interval (RRI), systolic blood pressure (SBP), diastolic blood pressure (DBP), and pulse pressure (PP). Key markers may be determined using software. In another embodiment, an un-calibrated surrogate for blood pressure may be used as the source for raw pulsatile waveforms.

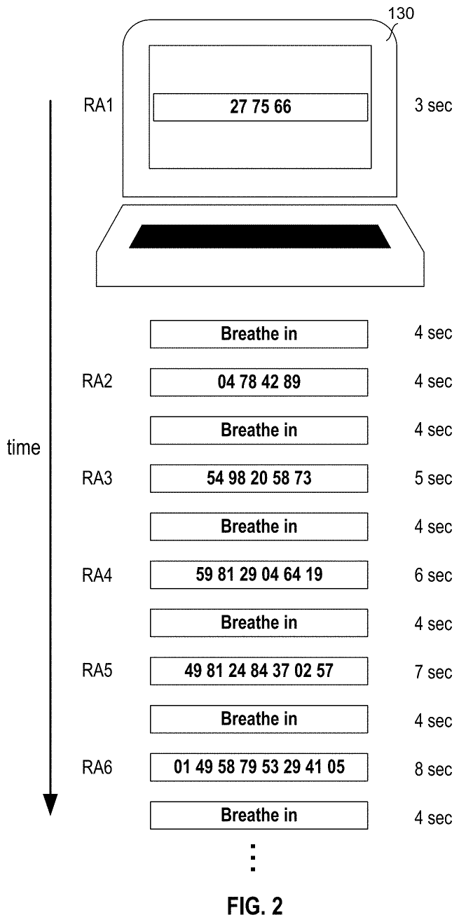

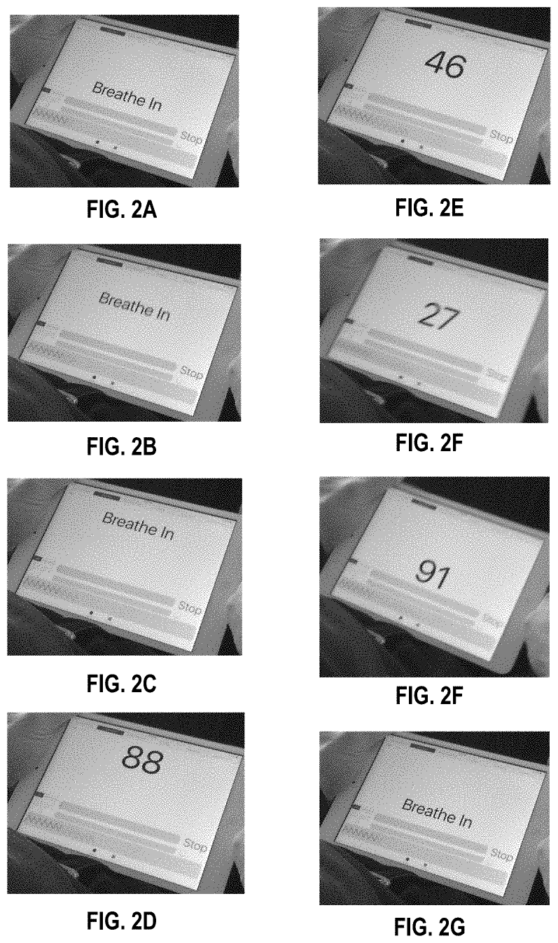

FIG. 2 is a representation of sets of information being displayed sequentially in time to be read aloud in a read aloud PADL session. Computer 130, which may be the computer from FIG. 1, is configured to display the sets of information sequentially in time. In this example, the PADL session involves displaying in an alternating manner a set of information to be read aloud and then displaying a message asking the individual to “breathe in”. The individual therefore has the opportunity to breathe in during the breathe-in intervals and exhale during the read aloud intervals. A pair of corresponding breathe-in and read aloud intervals is referred to as a read aloud cycle. Thus a read aloud interval is the time period during which the set of information corresponding to the given micro challenge is read aloud. In the example of FIG. 2, the first message displayed on computer 130 may be “breathe in” (not shown), which may be displayed for a predetermined time period, here 4 seconds. The next message displayed may be a first information set, here “27 75 66”, which is a set of three 2-digit numbers, as shown on computer 130 in FIG. 2. This first information set is labeled as “RA1” signifying that it corresponds to a first read aloud interval. The other information sets are similarly labeled. Computer 130 displays this information set for a predetermined amount of time suitable for allowing the individual to fully read the information set aloud. In this example, computer 130 displays the first information set for 3 seconds, which provides one second per 2-digit number. This information set is intended to serve as a physiological micro challenge to the human when read aloud.

Computer 130 then displays the “breathe in” message for 4 seconds. Next, a second information set “04 78 42 89” is displayed for 4 seconds. This information set is displayed a bit longer, here one additional second, relative to the previous information set to account for the additional 2-digit number to be read aloud. The addition of this additional 2-digit number is an increase in the size of the information to be read aloud compared to the previous information set, thereby increasing the intensity of the micro challenge of the second information set.

Computer 130 again displays the “breathe in” message for 4 seconds followed by a third information set “54 98 20 58 73” for 5 seconds. The breathe-in intervals and read aloud intervals continue to alternate as shown in FIG. 2.

The sequence of CV responses of the individual may terminate upon a first occurrence of a failure of one of the displayed sets of information to be read aloud. The termination may occur due to self-termination by the individual in response to the intensity of the one or more most recent micro challenges reaching an intolerable level in terms of comfort to the individual.

In an embodiment, the total duration of a PADL session, meaning from the first breathe-in message or first read aloud interval until self-termination, may be only a few minutes. As a mere example, a PADL session may last around 3 minutes. However, the duration may be a function or result of the intensity levels of the micro challenges and/or the CV health of the individual.

As the individual alternates between breathing in and reading aloud the information sets during the PADL session, a sequence of CV responses of the individual to each of the physiological micro challenges may be measured using the one or more sensors 110. Each of the CV responses is generally associated in time with the reading aloud of one of the sets of information and thus with a one of the micro challenges.

In an embodiment, the CV responses are transmitted to and stored at computer 130. Computer 130 may process the CV responses by comparing or otherwise analyzing at least one of the CV responses to predefined reference CV response information. The reference CV response information may be used in an assessment of the CV risk or CV performance of the individual. The reference CV response information may be predefined CV response data, for example established from data of a control group or designated healthy group of individuals. Additionally or alternatively, the reference CV response information may include data obtained for the same individual undergoing the current PADL session. For example, the reference data may be established from CV measurements taken at an earlier stage such as at or near the beginning of the PADL session.

An output of the comparison or analysis of the one or more CV responses of the individual may be a CV risk score or profile, and/or a CV performance score or profile.

In the example of FIG. 2, there is no particular significance to the particular 2-digit numbers or combination of 2-digit numbers that constitute each information set. They may be random. Furthermore, in an embodiment, on average the size of the information in each set of information increases over time thereby increasing the intensity of the micro challenges. In an embodiment, information sets do not increase in every subsequent information set displayed as they do in FIG. 2. For example, two or more consecutive information sets may be of the same or similar size. With reference to the example of FIG. 2, two or more consecutive information sets may have the same number of digits. In an embodiment, a subsequent information set may be smaller in size than a previously displayed information set.

Breathe-in intervals are not limited to 4 second durations. Rather, they may be any other suitable length in other embodiments.

Furthermore, in other embodiments, other types of information may be displayed, such as letters, words, numbers, pictures, drawings, shapes, etc. or any combination thereof. In other words, the present disclosure is not limited to displaying information sets consisting of numbers, including multiple 2-digit numbers.

FIGS. 2A-2G are screenshots of information being displayed sequentially in time to be read aloud in a read aloud PADL session according to another embodiment. In this embodiment, the individual is presented with real-time information about their current position within the current read aloud cycle. An example read aloud cycle is shown in FIGS. 2A-2G. In FIGS. 2A-2C, a “breathe in” message slowly rises on the screen to indicate to the individual how much of the breathe-in interval is remaining. The breathe-in interval ends when the “breathe in” message reaches the top of the display region on the screen. A read aloud interval then begins, as shown in FIG. 2D. The first two digit number “88” appears at the top of the display region. The two digits remain visible for 1 second as they slowly descend towards the bottom of the display region. After each second passes, a new two digit number appears to replace the previous digits and continue the descent towards the bottom of the display region. In FIG. 2E, new two digit number “27” has replaced number “88”. After another second passes, a new two digit number “91” appears to replace the previous digits “27” and continue the descent towards the bottom of the display region as shown in FIG. 2F. Thus, the height of the current two digit number represents the progress of the individual through the current read aloud cycle. The read aloud interval ends when the last two digit number reaches the bottom of the display region on the screen. FIG. 2F shows number “91” very close to the bottom of the display region. The individual may use this positional information of the numbers on the screen to govern the amount of air they use for reading aloud. This may prevent the subject from running out of respiratory volume before the end of the read aloud interval. Once the read aloud interval ends, a breathe-in interval begins and the “breathe in” message slowly rises again on the screen, as shown in FIG. 2G, to show the individual how much of the breathe-in interval is remaining. This real-time phase information helps the individual prepare for the next micro challenge increment by allowing them to govern the force and depth of their inspiration. The act of reading aloud itself causes the individual to expire at a fairly constant rate. In an embodiment, this is a very important factor for maintaining a consistent physiological context during expiration. The consistent physiological context reduces confounding influences (such as sporadic trans-thoracic pressure and downstream blood pressure changes), and facilitates the detection and measurement of subtle deviation features. The durations of the breathe-in and read aloud intervals in this embodiment may be similar or different than those of the embodiment of FIG. 2.

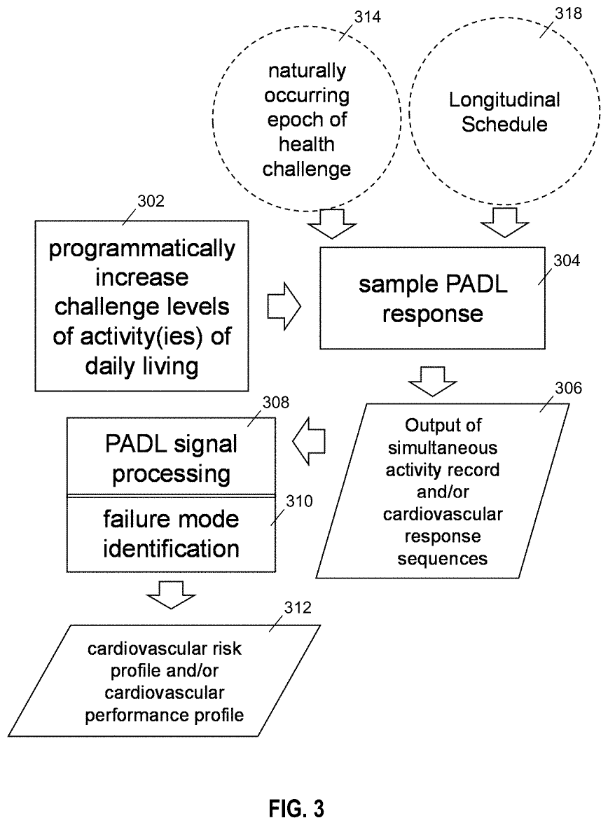

FIG. 3 is a process flow diagram of a general PADL method according to the present disclosure. The PADL may be any suitable type of PADL and is not limited to only read aloud PADLs. The process begins at block 302 by programmatically increasing challenge levels of one or more activities of daily living, as previously described. The process proceeds to block 304 where the response or responses of the individual to the PADL are sampled or otherwise obtained, for example at a computer. The process then proceeds to block 306 where data or other information representing the response of the individual to the PADL is outputted from the computer. The data may be in the form of an activity record and/or a CV response sequence. An activity record may be a record of what the individual was doing at that precise time of the recording. In an embodiment, a simple code is used to indicate exactly what the individual was supposed to be doing at that precise moment (for example, reading aloud the 2nd random number pair during the fourth reading aloud cycle). Thus the activity record may comprise a plurality of codes. This information is useful for performing precise signal processing relative to known physiological events in at least some embodiments. The process then proceeds to block 308 where the response data is processed, for example by comparing or otherwise analyzing the response data. The response data may be analyzed in relation to reference response data, for example as previously described. The process then proceeds to block 310 where an output of the processing done in block 308 may trigger a failure mode identification, which may represent that the response of the individual to the PADL is abnormal and potentially concerning. The process then proceeds to block 312 where a result of the processing done in block 308 and possibly a result of the identification done in block 310 are generated and outputted. The output may comprise a CV risk profile and/or a CV performance profile.

Additionally or alternatively, a naturally occurring epoch of health challenge 314, such as a temporary ailment, and/or a longitudinal schedule 318 may form part of the input to the PADL analysis, for example at sampling block 304. A naturally occurring epoch of health challenge may comprise, for example, a respiratory infection, a gout flare up, jet lag, temporary diarrhea or vomiting, or any other type of naturally occurring temporary epoch including inflammatory, infection or prolonged bed-rest, depression or temporary social/emotional interaction stress. In doing so, the PADL may reveal a preview of a future CV response deviation(s), which may only be revealed due to the additive macro challenge of the temporary ailment. A longitudinal schedule 318 is self-defined by the individual depending on their own emotional and/or physiologic context and it is self-actualized. Even so the person is prompted to do the analysis at least weekly and with any epoch of natural health challenge.

The generalized PADL method of FIG. 3 may be adapted to use a read aloud PADL. Thus, for example, block 302 would utilize a read aloud activity as the PADL.

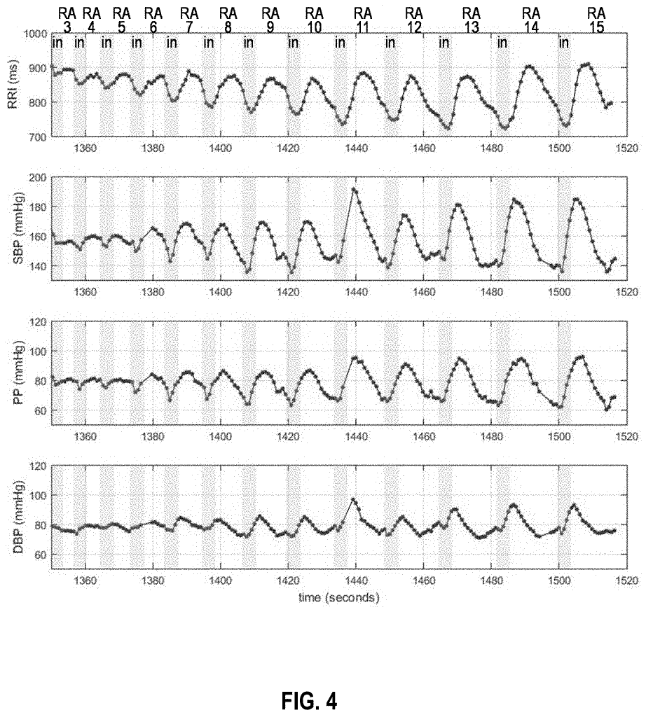

FIG. 4 shows a healthy CV response sequence to a read aloud PADL according to the present disclosure. The PADL may be the same PADL as described with reference to FIGS. 1 and 2. The CV response of FIG. 4 comprises four different CV parameters or measurements, namely R-R interval (RRI), systolic blood pressure (SBP), diastolic blood pressure (DBP), and pulse pressure (PP), which are plotted on four separate graphs. SBP refers to the systolic blood pressure measured for the current heartbeat. Likewise, PP and diastolic blood pressure DBP refer to the pulse pressure and diastolic blood pressure, respectively, as measured for each beat. Furthermore, it is to be appreciated that a CV response may include more, less, or differing CV parameters than those shown in FIG. 4.

Breathe-in intervals are indicated with shading in the graphs and are labeled “in” (for inspiration) while the intervening time periods are read aloud intervals and are labeled as “RA3”, “RA4”, “RA5”, etc. Similar to the PADL of FIG. 2, the durations of the breathe-in intervals are constant (e.g. 4 seconds) while the durations of the read aloud intervals gradually increase over time.

The CV response sequences of FIG. 4 were obtained from a designated healthy individual and illustrate a normal response to a read aloud PADL. In particular, the sequential increase in the duration of the read aloud intervals, and thus the increase in the micro challenges associated with each of the read aloud intervals, produced cyclic responses that generally increase in magnitude. This is seen in the increase of the amplitude of the line plots in each of the four graphs going from left to right as time progresses. Thus, the CV system of the individual generally responds to these small progressive increases in challenge levels. In the example of FIG. 4, the CV system of the individual responds with RRI, SBP, PP, and DBP cycles of progressively increasing magnitude. In this particular implementation of a PADL, the individual self-terminated the read aloud PADL based on self-determined discomfort.

Some abnormal CV responses to a PADL are now generally described. In an embodiment, the method or system is designed specifically to reveal any existing deviations, which may include subtle deviations, from a reference CV response such as a designated normal or designated healthy CV response. The reference CV response may include CV response data from a set of designated healthy individuals and in this regard may be considered a normal CV response. As previously mentioned, a deviation in a CV response generally refers to a CV response of an individual that differs from a reference CV response. A CV response may deviate from a reference response in one or more different ways and in terms of one or more CV parameters.

In an embodiment, a goal is to detect the earliest deviations from a designated healthy CV response. To do this, a read aloud PADL may gently increase the challenge level of a very simple activity of normal living, in this case reading aloud a string of numbers, to the point where first CV response deviations are detected. Often these earliest deviations are quite subtle. In an embodiment, the read aloud PADL is configured to limit the potential confounding influences that inhibit early detection. In an embodiment, a goal of read aloud PADL is to have sufficient sensitivity to longitudinally monitor subtle changes in the functional status of CV performance metrics, and/or also to monitor any subtle reduction or escalation of known CV response deviations.

Confounding influences are any physiological transients that are not timed to an external script. For example, in random sampling used by Holters, etc., the exact timing of inspiration and expiration, and the rates of inspiration and expiration are unknown, as are the state of the blood pressures and blood pressure baroreflex transients, and other skeletal muscle contractions. The unknown timings of these unknown transients can modulate all CV parameters at unknown times, thus the CV signals often appear to be influenced by pseudo-random factors. In at least some embodiments, PADLs are designed to prevent this; all the big physiological transients are synchronized and perfectly timed. The word “confounding” is used because the CV system is so integrated that small “un-accounted for” transient activities can produce measurable transient modulations of many CV parameters—thus they confound in the sense that it is not known what was exactly going on at that time. In an embodiment, PADLs are designed to minimize the unknowns. In fact, under PADLs, the transient unknowns are now the points of interest in at least some embodiments. For example, “why did SBP do that at that time”? With PADLs, the random confounding factors are purposefully prevented, thus, any newly detected deviation is likely to represent an invoked failure of the CV system.

In an embodiment, the individual is notified that they may self-terminate the PADL session when they become too uncomfortable in response to the increasing micro challenges. In an embodiment, the PADL is configured to be very easy at the beginning of the session so that the individual perceives or undergoes little or no physiological stress. This initial easiness prevents the individual from simply refusing to perform the PADL session because it is too difficult from the start. Furthermore, the easy beginning may provide a reference condition, which may be important in the analysis itself. Lastly, the easy beginning may initiate one or more cardio-vascular reflex cycles whose integrated physiological responses may extend into a next read aloud interval and/or breathe-in interval. Thus, in an embodiment, the read aloud PADL is purposely designed to be as gradual an increase in micro challenge level as possible within a relatively short, meaning practical, time frame.

It was observed in the responses from the individuals in the research study that there was substantial variance in the number of read aloud intervals until self-termination. For example, not every individual made it to a read aloud interval of 6 seconds in duration before self-termination. In an embodiment, it is presumed that at the beginning of the read aloud PADL session the individual is unstressed and self-termination by the individual marks the end of tolerable micro challenge. The analysis may be based on these two endpoints, namely no micro stress at the beginning of the PADL session, and subject-dependent intolerable micro stress at self-termination of the PADL session.

In an embodiment, the present method attempts to identify existing CV deviations falling into of one or more of the following categories of deviations. One category is where a new CV entity emerged during the read aloud PADL session. In other words, a new CV abnormality, for example a premature ventricular contraction (PVC) or a blood pressure alternan, manifested itself as a result of the increases in micro challenges on the individual.

Another category of deviation is one where a CV feature that normally cycles with every read aloud interval and breathe-in interval, such as blood pressure or heart rate, changes in a manner inconsistent with the normal cyclic changes established by a designated healthy control group or other reference information. For example, a common deviation is where one of the blood pressure measures, such as pulse pressure or heart rate, changes cyclically but the change is out of phase with the designated healthy cyclic variability.

Another category of deviation is one where a measured parameter changes incrementally over the span of the entire PADL session. For example, a common deviation is where heart rate or blood pressure increases towards the end of the read aloud PADL session.

In the research study, all of the observed deviations belonged to one or more of the above mentioned three categories of deviation. From a time perspective, each deviation is at a different scale: the first is at a heartbeat scale, the second is at the scale of the read aloud—breathe-in interval cyclicity, and the third category is assessed over the scale of the entire PADL session. It is to be appreciated, however, that some other deviations that may be detected may not necessarily fall into one of these three categories.

In an embodiment, read aloud PADL is configured for first detection of subtle CV deviations. Some individuals may experience some deviations from the start of a PADL session. For instance, some individuals with poor CV health will experience PVCs and/or alternans even during the initial easy read aloud intervals. Therefore the emergence of a new beat-scale CV entity is not a factor since the entity is already present and may be noted by the PADL system. If these sick individuals are then put on a drug treatment and/or exercise or oxygen supplementation treatment and if the treatment is successful, then read aloud PADL may eventually again detect that the treatment prevents the beat-based deviation under the zero-stress conditions at the beginning of read aloud PADL, only to be detected once the micro challenges have been incremented.

For some individuals, beat-based entities and/or the read aloud—breathe-in cyclic changes may deviate from normal during the read aloud cycle just prior to self-termination. This is a type of hybrid deviation because it involves multiple scales of deviation occurring just prior to self-termination. As an example, if an individual experiences SBP alternans early in the read aloud PADL (alternans are a beat-to-beat scale deviation), the magnitude and/or occurrence rate of the alternans often increases as the read aloud PADL continues to progressively increased micro stresses. Thus, the response deviation occurs at two scales—the beat-to beat scale (alternans) and the scale of the entire PADL (alternan modulation). Thus, this is a hybrid deviation because it occurs at two scales.

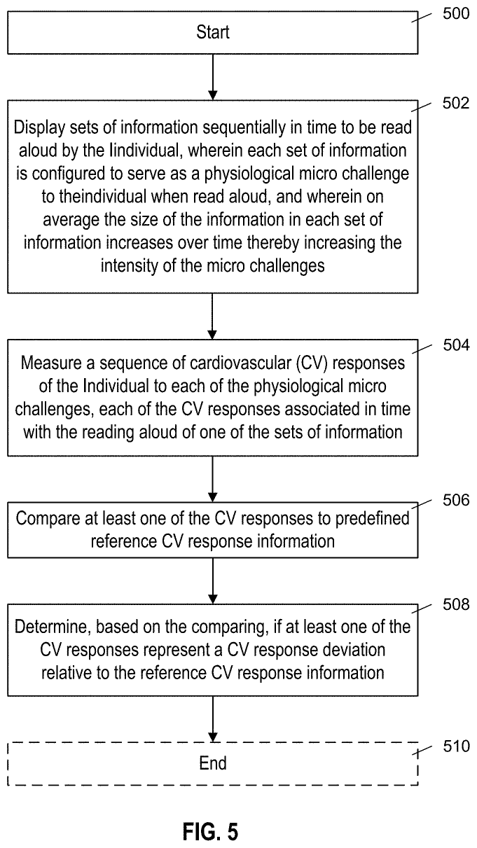

FIG. 5 is a process flow chart of an example method of a read aloud PADL according to the present disclosure. The method may be used in assessing the CV health performance or CV health risk of a human. The process starts at block 500 and proceeds to block 502 where sets of information are displayed sequentially in time to be read aloud by the human. Each set of information is configured to serve as a physiological micro challenge to the human when read aloud. On average, the size of the information in each set of information increases over time thereby increasing the intensity of the micro challenges. In an embodiment, the size of the information in each set increases on average over the duration of the PADL session.

The process then proceeds to block 504, where a sequence of CV responses of the human to each of the physiological micro challenges is measured. Each of the CV responses may be associated in time with the reading aloud of one of the sets of information.

The process proceeds to block 506, where at least one of the CV responses is compared to predefined reference CV response information.

The process proceeds to block 508, where a determination is made, based on the comparing, if at least one of the CV responses represents a CV response deviation relative to the reference CV response information.

The process may then proceed to block 510 and end.

In an embodiment, the determining may involve determining if one or more of the measured CV responses indicates a CV response deviation in the form of a heart beat scale CV abnormality that emerged during the sequence of measured CV responses. This type of CV abnormality is on a heartbeat scale since it may be identified by considering only a single heart beat cycle response of the individual. This is in contrast to other types of deviations or abnormalities that are typically only identifiable by examining a group of multiple heart beat cycles. A CV response may be abnormal relative to a reference heart beat scale CV response in the reference CV response information. Such an abnormal CV response is considered a CV abnormality. Examples of heart beat scale CV abnormalities include but are not limited to PVCs, and BP alternans. Technically, alternans are a couplet of beat responses: one larger beat response followed by one lesser beat response. However, for these purposes, alternans may be considered a heart beat scale CV abnormality in at least some embodiments.

In an embodiment, the measuring of a CV response to a given physiological micro challenge may involve measuring the CV response spanning a read aloud interval. The determining may involve determining if there is a CV response deviation in the form of an abnormality in the way in which the read aloud interval CV responses of the human change during the sequence of measured CV responses. This type of CV abnormality is on a scale of read aloud interval breathe-in cyclicity since it may be identified by considering multiple read aloud cycles of the individual in order reveal a change between cycles. This is in contrast to types of deviations or abnormalities that may be identified by examining a single heart beat cycle, or by examining most or all of the CV responses in the entire PADL session. A change in the read aloud interval CV responses may be abnormal relative to a reference change in read aloud interval CV responses. Such an abnormal change may be considered a cyclic change abnormality. Examples of cyclic change abnormalities include but are not limited to a blood pressure change, a heart rate change, or any other read aloud interval CV response that changes in a manner partly or completely inconsistent with normal cyclic changes represented in the reference change information, for example designated normal cyclic changes established by a designated healthy control group.

In an embodiment, the determining may involve determining if there is a CV response deviation in the form of an average incremental change in a CV characteristic during the sequence of measured CV responses that is abnormal relative to reference change information for the CV characteristic. Here, the sequence of measured CV responses are all of the CV responses of the individual during the PADL session. This type of CV abnormality is on a scale of a PADL session since it may be identified by considering most or all of the CV responses in the entire PADL session. This is in contrast to types of deviations or abnormalities that may be identified by examining a single heart beat cycle, or by examining multiple read aloud cycles without necessarily examining all cycles within the PADL session.

Some abnormal CV responses are described in more detail.

One type of deviation that is commonly observed during a read aloud PADL is the spontaneous de novo appearance of a new CV entity at some unforeseen point in the read aloud PADL. The de novo CV entity is only new with respect to the responses recorded for the current individual undergoing read aloud PADL. Many of the 350 subjects who performed read aloud PADL in the research study showed the same de novo CV entities emerging at some latter stage of read aloud PADL. Often, the initial manifestation of a de novo CV entity is very subtle. In an embodiment, a design philosophy for a read aloud PADL was to detect these entities at the earliest possible instance, before the entity itself can increase in physiological significance and spawn other CV complications. Only a few of the 350 subjects manifested some of the known CV entities right at the lowest challenge start level of read aloud PADL. For these subjects, the entities were not de novo with respect to read aloud PADL. Nevertheless, the deviant CV entities were detected, and their magnitude and occurrence rates may be accurately monitored using read aloud PADL. The 350 test subjects did manifest several de novo cardiovascular entities commonly known to cardiology. However, because read aloud PADL is new and is designed, in at least some embodiments, for reproducibly detecting subtle de novo deviant entities, it is highly likely that there are many other hitherto unknown subtle CV entities that may be detected by read aloud PADL. Future discovered entities would likely depend on the vastly different patient populations that use read aloud PADL. In this sense, read aloud PADL may be a home-based platform that may be used for the discovery, detection and monitoring of hitherto unknown cardiovascular entities.

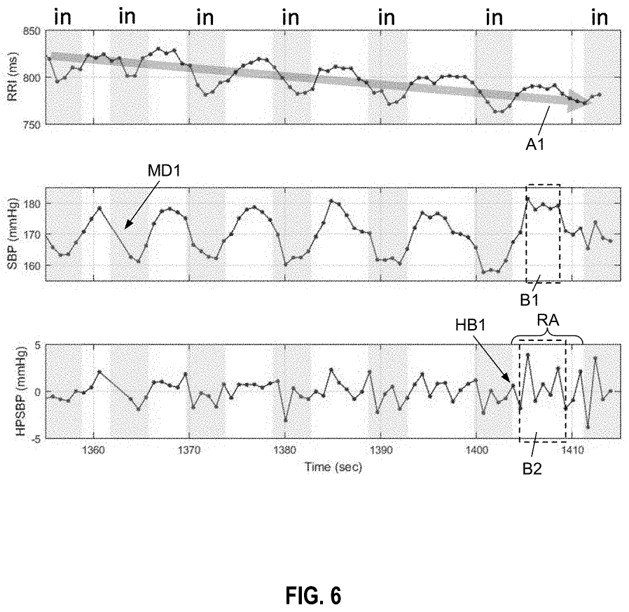

In particular, a type of deviation is a de novo read aloud PADL induced SBP alternans. FIG. 6 is a set of graphs showing CV response sequences to a read aloud PADL that reveal the emergence of a SBP alternans deviation. Two different CV parameters, namely R-R interval (RRI) and systolic blood pressure (SBP), are plotted on two separate graphs. A third graph represents high pass filtered diastolic blood pressure (HPDBP), meaning the DBP response data that has been put through a high pass filter.

The RRI graph shows that SBP alternans occur independently of simultaneous RRI alternans. The alternan sequence in the shaded box labeled B2 of the HPSBP graph is detected in the high-pass filtered SBP sequence. Note that the alternan sequence within the HPSBP signal, indicated by the broken line box labeled B2, contains 7 alternan beats. The unfiltered SBP alternan sequence indicated by the broken line box labeled B1 in the SBP graph contains only 5 beats. The two missing alternan beats in the unfiltered SBP subsequence have been masked by steeper BP slope changes associated with the slowly changing BP cyclic waveform. These slower cycles are associated with baroreflex BP changes; these changes are usually band limited to a low frequency such as 0.1 Hz. Thus, in an embodiment, read aloud PADL uses a high-pass digital filter to eliminate any potential baroreflex related BP changes.

In an embodiment, the definition of alternans is a slightly refined version of alternans as defined by the community. In an embodiment, alternans is defined as any contiguous sequence of beats, at least 3 beats in length, wherein the individual beats sequentially alternate between local minimum beats and local maximum beats. In math, these local minima and local maxima are referred to as “turning points”, where the slope of the sequence or function changes sign. Thus, alternan detection may be applied to any contiguous beat sequence (which is at least 5 beats long—need 5 beats to find 3 consecutive turning points) such as RRI, SBP, DBP, PP, etc.

As mentioned earlier, the CV system is highly integrated. In at least some embodiments, this high level of integration may also affect the detection of blood pressure alternans.

FIG. 6 illustrates two common things which can affect the detection of BP alternans. In the SBP graph (middle graph), SBP is being cyclically modulated in phase, with respect to the “breath-in/read aloud” cycles. This is caused by cyclic baroreflex arterial contraction (high pressure) and arterial dilation (low pressure), and this cyclicity is invoked by strong transient transthoracic pressures set up by the deep cyclic phasic respiration of the read aloud PADL. Unfortunately, this slow baroreflex cyclicity is of high magnitude, so high that the small magnitude beat-to-beat SBP alternans are swamped by the larger SBP transients experienced when SBP is increasing or decreasing. This is why the slow cycles of the background are filtered out. The two BP modulations are superimposed—alternans and baroreflex, and the baroreflex modulation can be much larger in magnitude and can swamp the alternans in superposition.

Another facet of this high degree of CV integration is the dependence of the current BP value on the length of the filling-time immediately preceding the beat's contraction. If the heart has had a longer time to fill during the current cardiac cycle, then there is more blood in the chambers, and the resulting contraction will cause increased pulse pressure for the ensuing beat. So, if the filling-times of the heart are manifesting an alternan pattern due to some deviant sinus node condition (not uncommon), then this filling-time alternan pattern can produce BP alternans whose origins are simply due to artifacts of the alternan pattern in filling-time. This is quite common in the data set from the research study mentioned previously. Thus, these artifactual alternans can confound the search for all blood pressure alternans. Moreover, these artifactual alternans do not have the same origin as the other SBP alternans that were observed, and they are quite common in otherwise healthy people. The designated pathological blood pressure alternans are not due to this artifact of filling-time. This is why there is an attempt to try to isolate those blood pressure alternans that are independent of alternans in filling-time, (using RR-interval as a proxy for filling-time). Thus, for FIG. 6, there are no alternans in RRI for the last read aloud interval. Therefore, the measured simultaneous SBP alternans of FIG. 6 are not artifactual in origin, thus they may represent the real pathological blood pressure alternans that are sought. That is why BP alternans are sought independently of RRI alternans. In at least some embodiments, this is a very important step. All methods seeking to detect designated pathological BP alternans would have to account for this. Otherwise they would be mixing pathological and artifactual BP alternans and this would be highly inaccurate.

FIG. 6 also shows that the alternan subsequence in the HPSBP sequence indicated by box B2 has excluded the first heartbeat, labeled HB1, of the corresponding read aloud interval, labeled RA. Such “first” reading aloud beats of read aloud intervals often have erratic BP expression due to the effects of rapid changes in intra-thoracic pressure caused by respiratory phase change immediately preceding the contraction of the beat. Also, heartbeats during breathe-in intervals may be excluded from the alternan search. Breathe-in intervals are shown in shaded boxes and are also labeled “in” in the top RRI graph. Each breathe-in interval in this example is brief (4 seconds) and the inspiration force increases as the read aloud intervals incrementally increase in duration. This is quite unlike the controlled expiration rates of the read aloud intervals and for this reason the breathe-in beats may be excluded from the alternan search.

Still referring to FIG. 6, processing of the CV response of the individual in an embodiment is now described. Signal processing of the CV response data may begin with linear interpolation. In this embodiment, the SBP data were acquired using a 1 kHz sampling rate. Very often, there may be gaps in the data where the SBP data is missing, usually due to a momentary calibration run of the sensor, in this case a BP measuring device, or intermittently by bad data caused by motion artifact. An example of missing data is indicated by arrow MD1 in FIG. 6. In this case, there are several missing beats due to a brief auto-calibration of the BP measuring device. As well, there are often PVC or atrial premature complexes (APC) beats within the SBP response recording. In this embodiment, the BP values for these non-sinus beats are excluded for all alternans searches. The remaining gaps are also filled by linear interpolation. In detail, a first signal-processing step is to linearly interpolate all sinus beat SBP values to a constant time spacing. In keeping with the 1 kHz sampling, all SBP values are linearly interpolated to a 1 ms time grid. Next, the interpolated SBP data is filtered using a finite impulse response (FIR) filter designed via the Hamming window method using 20000 filter coefficients, and a cut-off frequency of 0.1 Hz. The digital filtering is performed in both time directions to eliminate any lag. As well, the Hamming window FIR does not produce any spurious “ringing” which would greatly confound the search for subtle alternans. Finally, the high-pass filtered SBP data obtained at each SBP time index is used to yield the high-pass filtered sequence.

A CV response may be compared to a predefined reference CV response, in at least some embodiments, as follows.

In essence, if some deviation, such as alternans, occurs later in the protocol and it is not there early in the protocol then something has happened. It may be determined if there are statistically more alternans at the end of the PADL protocol, relative to the beginning of the PADL protocol. A sequence of CV responses may be divided into two subsequences—the first subsequence is the subsequence of beats in the read aloud PADL response from the beginning beat to the last beat before the first occurrence of an alternan; and the second subsequence is the subsequence from the first occurrence of the alternan to the last beat of the PADL response. A Chi-squared statistical comparison may then be performed on the two groups to determine if there is a statistically significant difference between these groups.

This raises another aspect of the PADL approach in general according to the present disclosure. For sparse entities such as de novo alternans, the individual often self-terminates shortly after the first cycle of occurrence. Thus, this would normally provide a weak statistical position because there are so few deviant events for the Chi-square comparison. But, because the same PADL test is being performed several times, for example every day, and in the same way, the data from the same phases of the read aloud PADL may be lumped together and analyzed. In at least some instances, the more often the PADL is performed, the more accurate the comparisons become because more statistical power is gained by performing the identical PADL. Thus, for example, the first 3 cycles of PADL may be compared, with the last 4 cycles. A comparison may be performed in any other suitable way, meaning not limited to comparing the first 3 read aloud cycles to the last 4 read aloud cycles. All the data from a similar physiological context may be compiled in this fashion. This type of signal processing may be referred to as “contextual signal processing”. In at least some embodiments, it provides an advantage over random sampling because these kinds of contextual comparisons are performed.

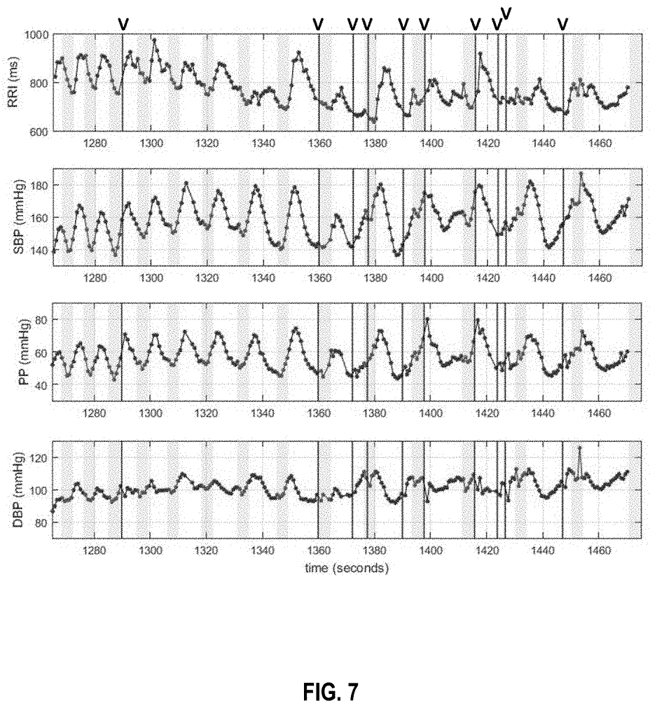

Another type of deviation is a de novo emergence of premature ventricular contractions (PVCs) and/or atrial premature complexes (APCs). FIG. 7 shows a read aloud PADL CV response with isolated PVCs, labeled “v”, emerging in the latter half of the CV response. Similar to FIG. 4, in FIG. 7 four different CV parameters, namely RRI, SBP, DBP, and PP, are plotted on four separate graphs.

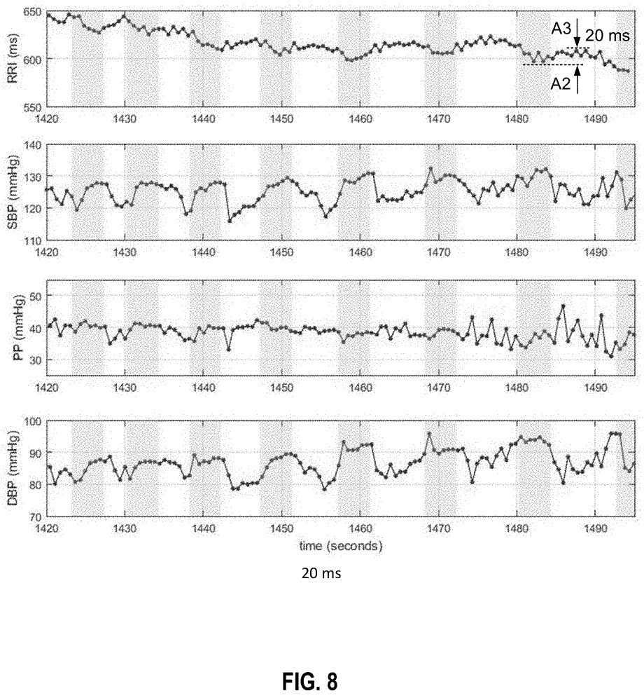

Another type of deviation is a lack of inspiratory relative tachycardia and loss of expiratory relative bradycardia that creates a metric of lack of bradycardic capacity. FIG. 8 is a set of graphs showing CV response sequences to a read aloud PADL that reveal a lack of bradycardic capacity deviation. The RRI graph in FIG. 8 shows very little change in RRI between an RRI minimum during a breathe-in interval, indicated by the broken line indicated by arrow A2, and a maximum RRI during a read aloud interval that immediately follows, indicated by the broken line indicated by arrow A3. The bradycardic capacity may be determined by the average difference between the inspiration minimum RRI and the ensuing reading-aloud maximum RRI.

Another type of deviation is relative and progressive tachycardia during the entire read aloud PADL. FIG. 6 shows a typical progressive decrease in RRI, indicated by the large arrow labeled A1 in the RRI graph induced during the read aloud PADL session. A linear regression of RRI as a function of time produces the average slope of the progressive relative tachycardia.

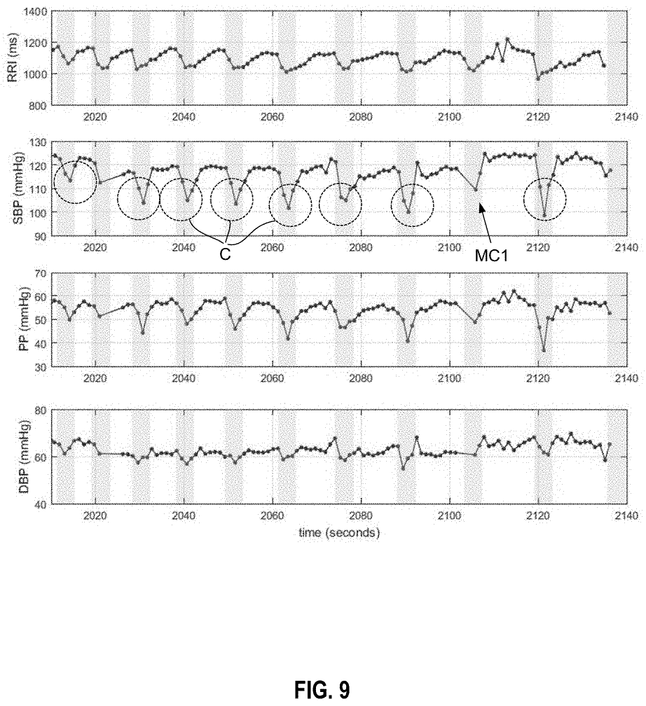

Another type of deviation is a sharp inspiratory SBP drop and subsequent recovery within a prescribed number of breathe-in beats. FIG. 9 is a set of graphs showing CV response sequences to a read aloud PADL that reveal a sharp inspiratory SBP drop deviation. The SBP graph in FIG. 9 includes several broken line circles C each indicating three beats that define a SBP minima during each breathe-in interval. The magnitude of the sharpness is defined as the average of the magnitude of the single beat SBP drop and the subsequent single beat increase that follows. At latter stages of the read aloud PADL session, the inspirations by the individual require larger volumes of air-flow effort to sustain the ensuing read aloud interval. Thus, the latter stage inspirations are more forceful. For this reason, each inspiration SBP sharpness may be normalized by dividing by the number of seconds of the ensuing read aloud interval. The measured value is the average of these normalized values. In the SBP graph in FIG. 9, the second-to-last inspiration SBP drop (note the missing broken line circle, labeled with arrow MC1) is not defined due to insufficient number of beats. In this case, an automated BP calibration run caused several beats to be unmeasured. As well, this measure uses only sinus beats during inspiration. Thus, PVCs and or APCs may also result in the loss of an inspiratory sharpness measure for any particular inspiration interval.

As previously described, a deviation in a CV response generally refers to a CV response of an individual that differs from a reference CV response such as a designated healthy CV response; and further that a CV response may deviate from a reference response in one or more different ways and in terms of one or more CV parameters. The following relates to various definitions of a deviation.

During the previously mentioned research study, during which the CV responses to read aloud PADL for the 350 or so subjects were observed, many things that were different in the sick subjects relative to the designated healthy group were seen. These differences seem to belong to one of the following types:

(1) The occurrence of an entity of CV response behavior not found in the set of designate healthy CV responses. The entity is some sort of organization defined at the beat scale, breathe-in/read aloud cycle scale, or at the scale of the entire read aloud PADL. This new entity of behavior, not found within the set of designated healthy CV responses, is defined as some sort of new unhealthy behavior of the CV system in response to the read aloud PADL micro challenges. (2) The absence of a designated healthy CV response behavior entity. (3) The abnormal alteration of a normal CV response behavior entity. Of these three types of differences, the first two are generally the easiest to define; the response behavior entity is either there or not there. The third type of difference is by far the most complicated to define. There are many ways that a CV response behavior entity can be altered. In general, some behavior entities represent a healthier state when they are larger in magnitude (such as bradycardic capacity), and some behavior entities are healthier when they are reduced in magnitude (such as transient reduction in blood pressure during inspiration). Moreover, many normal behavior entities display other aspects of measurable organization that also indicate trends toward healthy versus unhealthy. For example, if the blood pressure responses to read aloud PADL are “smooth”—as can be measured by a lack of spectral power at higher frequencies, then this individual is trending towards healthy, relative to an individual with a lot of spectral power at higher frequencies. Again, only the designated healthy versus unhealthy trends are known for the known abnormal alterations in designated healthy CV response behavior entities. The exact thresholds where alterations become significant “deviations” are presently unknown.

Likewise, only the sicker versus less sick measurable trends may be known for those occurrences of new unhealthy CV response behavior entities of type 1. For example, more frequent occurrences of SBP alternans is generally an indication of increased sickness levels. As well, larger magnitude SBP alternans are generally a sign of a sicker condition. But again, only these trends may be known, and not necessarily the threshold values that indicate imminent risk of serious CV event. Scientifically, individuals could be tracked for many years until they experience some sort of significant CV event.

According to an aspect of the present disclosure, techniques are provided for yielding measurable quantities, such as read aloud CV response behavior entities, whose presence and/or absence, and whose measured value is indicative of relative health. Thus, in this way, read aloud PADL may be compared to a home blood pressure machine—it yields some measured physiological values whose interpretation can indicate relative levels of health. Except that read aloud PADL, in an embodiment, provides a suite or profile of more specific measurable parameters that may be monitored over time (meaning longitudinally) to indicate worsening or improving health status, and thus increasing and decreasing CV risk, respectively.

In at least some embodiments, the emergence of new deviation response behavior entity does not require a threshold; nor does the disappearance of a normal healthy response behavior entity—these emergence and disappearances are significant. However, some or all measurable response behavior entities, either normal or deviant, can yield a number indicating one aspect of relative health/risk status for the individual. In some cases, the absolute significance thresholds for any of these individual measures is generally unknown, due to the diversity of personal health conditions and the paucity of data. A single CV risk/performance score may be created from the weighted contribution of the various measured response behavior entities, and that this combined score may be more effective at assessing relative CV risk/performance.

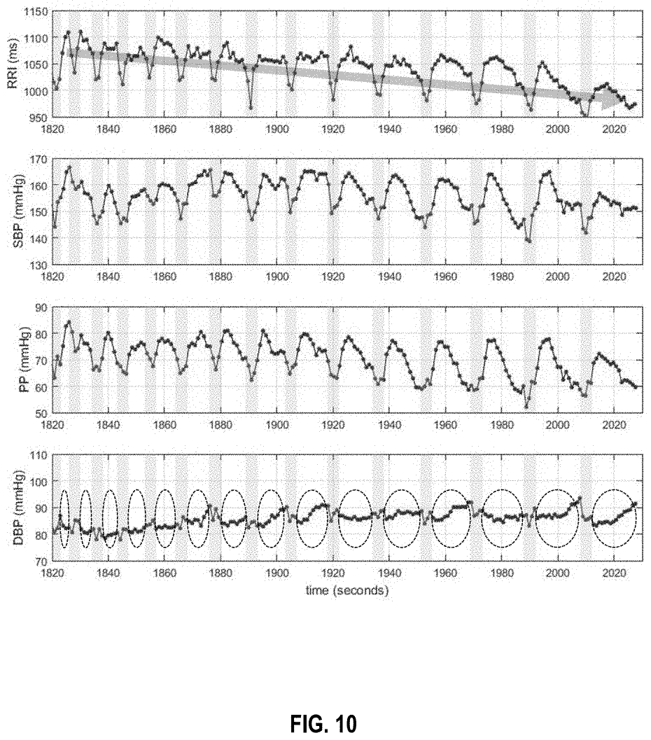

Another type of deviation is a failure in one or more of DBP, PP, and SBP to modulate in response to a read aloud PADL session. FIG. 10 is a set of graphs showing CV response sequences to a read aloud PADL that reveal a deviation in the form of a failure of the DBP to significantly or notably change in response to the read aloud PADL session. The top graph in FIG. 10, showing RRI, shows progressive relative tachycardia. In particular, the DBP graph in FIG. 10 shows the failure of the DBP to significantly change in magnitude and in phase in response to the read aloud PADL. The regions indicated by the broken line ovals in the DBP graph indicate the absence of a cyclic DBP response. In this embodiment, the determination is made by calculating the average BP value for all beats during the breathe-in intervals, and the average BP value for all beats during the read aloud intervals. In other words, a single average BP value is calculated for beats in all breathe-in intervals for the entire PADL session. Similarly, a single average BP value is calculated for beats in all read aloud intervals for the entire PADL session. In this embodiment, the same rules apply for all blood pressure sequences, meaning SBP, PP, and DBP. If the read aloud average BP value is not significantly greater than the breathe-in average BP value then this may be considered a BP response deviation. BP modulation deviation from designated normal may result from a lack of significant BP change and/or a poor phase-relationship of the particular BP response to the read aloud PADL.

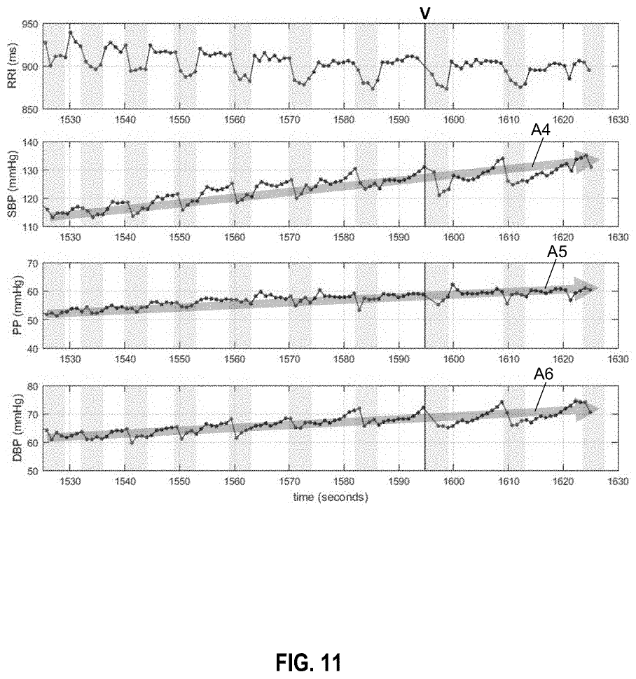

Another type of deviation is a progressive hypertension in DBP, PP or SBP in response to a read aloud PADL session. FIG. 11 illustrates a CV response having BP responses having progressive hypertension as indicated by arrows A4, A5 and A6 in the SBP, PP, and DBP graphs of FIG. 10, respectively. Note that in FIG. 10, the measured SBP beat response is simply equal to the DBP plus the PP for any given beat. Thus, in this case, the progressive hypertension in SBP has a slope equal to simply the sum of the slopes for DBP and PP. In this embodiment, the slopes are determined by the linear regression of the sinus BP beat values as a function of time (seconds). Also note the single PVC occurrence indicated by a vertical line in all four graphs and marked by a “v”.

Some guidelines or rules for correctly identifying BP alternans are now described.

A CV response to a read aloud PADL session may consist of sequences of individual CV measurement values, such as RRI, DBP, PP, and SBP, defined on a beat basis, meaning one measured value per beat. By definition, alternans subsequences are contiguous subsequences wherein the beat-based CV measured values sequentially alternate between local minimum and local maximum for some finite number of beats. Many transient physiological factors can affect the magnitude of a CV measured value. Thus many transient physiological factors can affect the search for subtle pathological alternans.

In an embodiment, read aloud PADL is configured to find only those BP alternans caused by pathological alternan modification of normal sinus contraction. These pathological BP alternans are distinct from the apparent BP alternans that can be generated by other non-pathological respiratory and CV processes.

One or more of the following techniques may be used in a read aloud PADL session to limit the potential confounding influences that might mask any subtle pathological alternans.

One technique is to use only normal conducting sinus beats. For individuals not experiencing atrial fib/flutter, a read aloud PADL may search for alternans only within subsequences of normal conducting sinus beats. PVC and APC beats have varying blood pressure manifestations due to their irregular contraction timing and mechanics.

Another technique is to use only beats from the read aloud interval. Breathing directly affects BP via changes in intra-thoracic pressure. In particular, changes in breathing direction may have an immediate effect on the BP of the succeeding beat. Thus, a read aloud PADL may exclude the first sinus beat of the read aloud interval, and search for alternans only within those remaining sinus beats that occur during the read aloud intervals. The physiological process of reading aloud maintains a steady airflow during expiration, meaning during a read aloud interval, and thus creates a very steady respiratory dynamic. This reduces the potential confounding influences of respiration in the search for alternans. Moreover, the read aloud interval progressively increases in duration thus providing longer subsequences (more beats) for detecting alternans.

Another technique is to isolate BP alternans independent of RRI alternans. A CV response to a read aloud PADL session often shows BP alternans that occur simultaneously with RRI alternans. This may be quite common in CV responses to a read aloud PADL. In these cases, the origin of the BP alternans may be completely or partially caused by the RRI alternans. Longer RRI times lead to longer filling-times that generally result in lower DBP values, and greater PP values for the given beat. To eliminate this potential confounding source of BP alternans, an embodiment of the read aloud PADL searches for only those BP alternans that are independent of simultaneous RRI alternans.

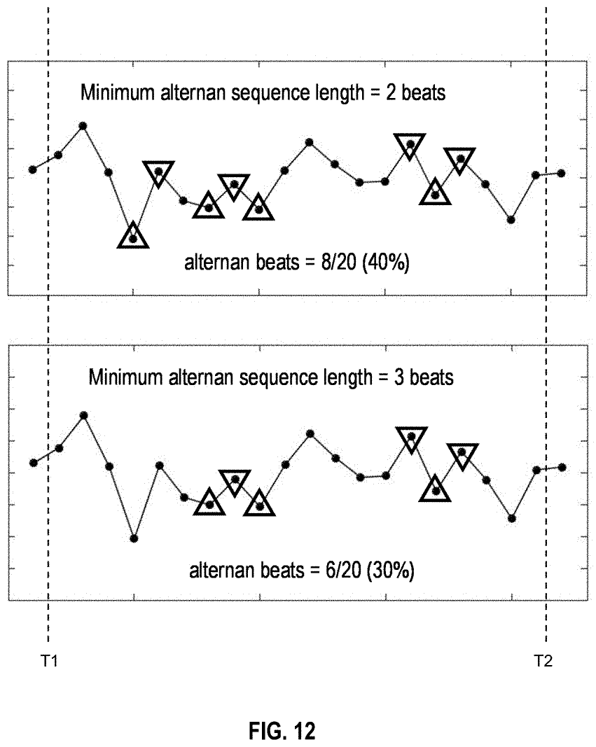

Another technique is to use minimum 3-beat alternan sequence length. Response subsequences of BP alternans can vary substantially in beat length. In an embodiment, the present read aloud PADL searches for alternan subsequences that are at least 3 beats in length; either min-max-min, or max-min-max. The 3 beat minimum length is a compromise. Based on some mathematical modeling, for identical sequences of Gaussian random noise, 57% of the data points occur in alternan subsequences that are at least 2-samples in length. This decreases to 46% if the minimum alternan length is increased to 3-samples. Thus, the requirement of 3 successive alternan beats substantially reduces the chance that an identified alternan subsequence is simply due to random fluctuations only. Obviously increasing the minimum length to 4, 5, and 6 beats would continually decreases the random origin possibility, but these longer contiguous beat lengths also demand longer read aloud expiration times that are free from PVCs and APCs. Thus, this embodiment of the read aloud PADL uses a compromised minimum alternan subsequence length of 3 beats. A comparison of delineated alternan subsequences using minimum runs of 2 beats vs. 3 beats is shown in FIG. 12. The graphs in FIG. 12 were generated based on some mathematical modeling and are provided as an illustrative example. For the given random sequence shown, the 2-beat minimum yields 40% alternan beats, whereas the 3-beat minimum yields only 30% beats during the same time period, as indicated by vertical broken lines T1 and T2. In another embodiment, the beat minimum length may be 2 beats.

The identification of CV response deviations in individuals with atrial fibrillation/atrial flutter (AF) using a read aloud PADL is now described.

Many of the known CV response deviations, including those described above, may also be detected in CV response data obtained from individuals with atrial fibrillation/atrial flutter (AF). However, the random cycle-length characteristic of AF can contribute to considerable high-frequency beat-to-beat variation in the RRI and BP responses.

The known deviations in AF individuals more or less parallel the previously documented known deviations for non-AF individuals. However, in at least some circumstances, the effect of the random cycle length inherent in AF individuals benefits from modification of one or more of the present techniques and/or complete elimination of other known response deviations from consideration during a PADL session analysis. Some modifications are described below, and are described in the same order as the deviations known to occur in non-AF individuals described above. Again, deviations may occur in isolation or they can be simultaneous.

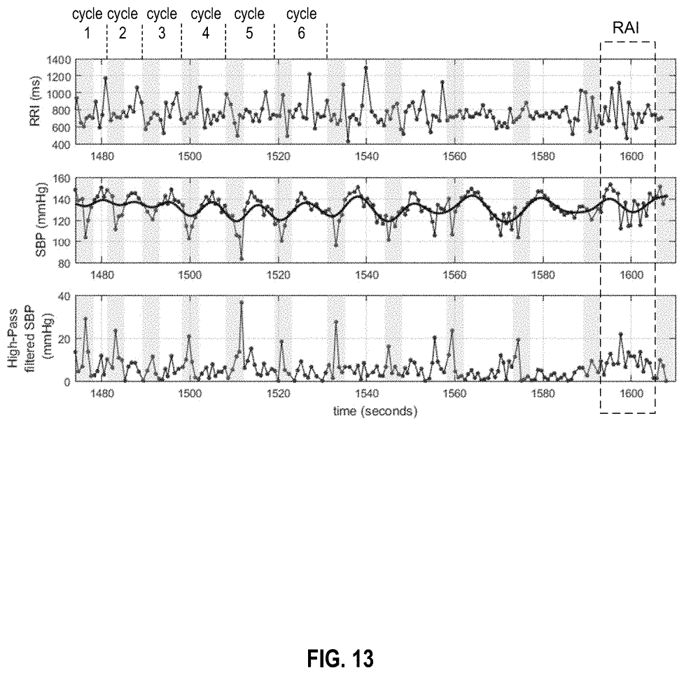

In terms of de novo SBP alternans, the random cycle lengths of AF are often associated with random beat-to-beat fluctuations of SBP. This often manifests as AF-generated SBP alternans randomly distributed throughout the read aloud PADL SBP response. An example is shown in FIG. 13. Thus, it may not be useful to detect the emergence of these random AF related SBP alternans. However, as shown in FIG. 13, the magnitude of the SBP alternans increases drastically and unexpectedly towards the end of the read aloud PADL session as indicated by broken line box labeled RAI. In particular, the SBP alternans during the last read aloud interval RAI of the read aloud PADL session are markedly increased in magnitude relative to all previous SBP alternans. As seen in FIG. 13, the SBP response includes a component of low-frequency SBP changes associated with baroreflex activity. To assess the true magnitude of the SBP alternans, in an embodiment the SBP response is high-pass filtered at 0.1 Hz to isolate the alternan activity. FIG. 13 shows the magnitude of the high-pass filtered SBP response. Although the high pass filtering frequency of 0.1 Hz was used, any other suitable low frequency may be used.

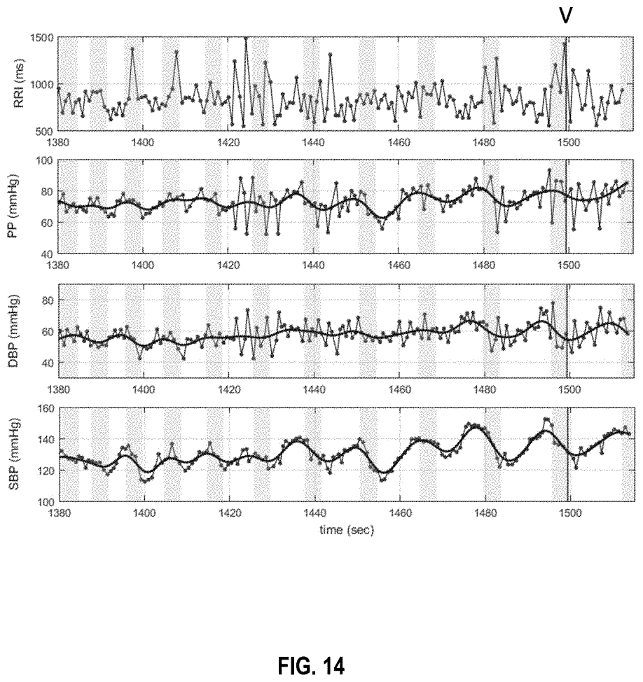

In terms of de novo emergence of premature ventricular contractions (PVCs), FIG. 14 shows a CV response to a read aloud PADL of an AF individual. The vertical line labeled “V” in the latter half of the recorded CV response corresponds to a late PVC. APCs cannot be identified in AF, thus this deviation only applies to PVCs for AF individuals.

In terms of lack of inspiratory relative tachycardia and loss of expiratory relative bradycardia that creates lack of bradycardic capacity, the absence of sinus beats during AF precludes the search for this specific deviation in AF individuals.

In terms of lack of progressive relative tachycardia, again, the absence of sinus beats during AF precludes the search for this deviation in AF individuals.

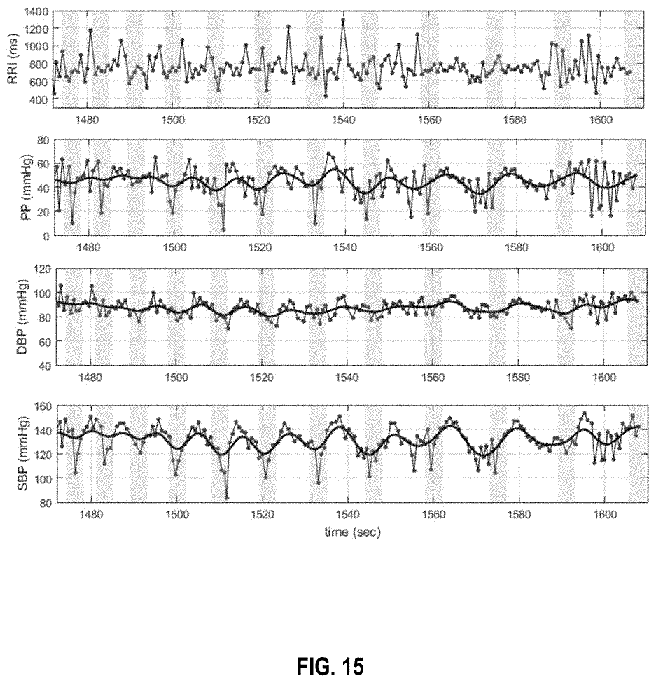

In terms of sharp inspiratory SBP drop and recovery within a prescribed number of inspiratory beats, AF causes increased variance in the beat-to-beat BP responses. In particular, during inspiration, the BP response to inspiration can be complicated. For some inspirations during breathe-in intervals, the sharpness of the local BP minimum may get exaggerated due to local AF conditions, while for other inspirations the sharpness might be severely attenuated. For this reason, the method for quantifying local inspiration BP drop sharpness is modified in an embodiment to work for AF individuals. In this embodiment, the magnitude of the sharpness is defined as the greatest average SBP difference of three consecutive SBP beats measured relative to the low-pass (e.g., 0.125 Hz) filtered SBP minimum occurring during inspiration. In other embodiments, a different low-pass frequency and/or number consecutive SBP beats may be used. As with the non-AF individuals, each inspiration SBP sharpness may be normalized by dividing by the number of seconds of the ensuing read aloud interval. The measured value may be the average of these normalized values. PVC beats are excluded from this analysis. This may result in the loss of an inspiratory sharpness measure for any particular breathe-in interval. FIG. 15 shows the inspiration minima relative to the low-pass filtered BP signals in an example CV response. The low-pass filtered BP signals are indicated by the smoothed curved lines in the PP, DBP and SBP graphs of FIG. 15.

In terms of failure in DBP, PP and/or SBP modulation in response to read aloud PADL, FIGS. 14 and 15 show a BP response to a read aloud PADL session for AF individuals. The responses show varying degrees of high-frequency AF beat-to-beat randomness superimposed on a slowly changing BP background signal related to baroreflex. This superimposed randomness causes an increase in measured BP variance. Thus this method generally requires additional signal processing for use within the AF population. The smooth, thick curve in each of the PP, DBP, and SBP graphs in FIGS. 14 and 15 is the matching low-pass (<0.125 Hz) filtered BP response. This measure is calculated by determining the average filtered BP value for all breathe-in beats and the average filtered BP value for all read aloud beats. These filtered BP beat values are obtained from the low-pass filtered BP signals measured at the time of the systolic pressure of each beat. If the read aloud average BP is not significantly greater than the inspiration average BP value, then in an embodiment this is considered a BP response deviation. BP modulation deviation from designated normal may result from a lack of significant BP change and/or a poor phase relationship of the particular BP response to the read aloud PADL.

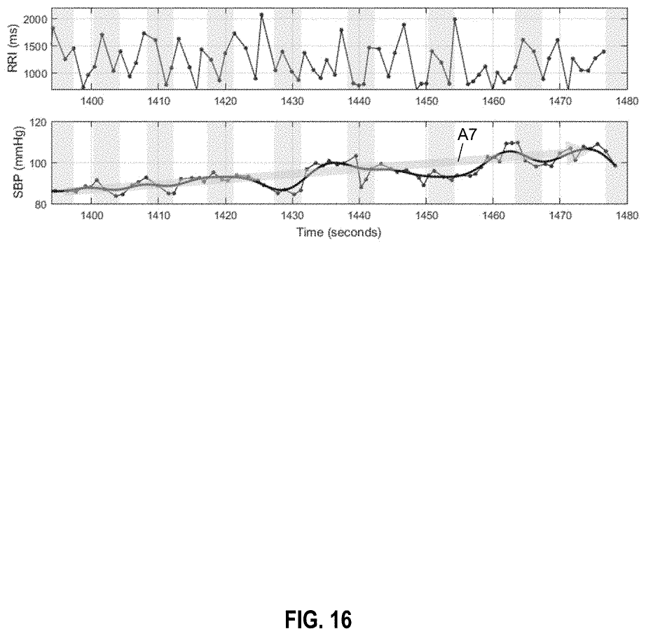

In terms of progressive hypertension in DBP, PP or SBP in response to a read aloud PADL, FIG. 16 illustrates a SBP response in an AF individual that has progressive SBP hypertension, as indicated by arrow labeled A7. The smooth thick curve is the matching low-pass (<0.125 Hz) filtered BP response. These filtered BP beat values are obtained from the low-pass filtered BP signals measured at the time of DBP or SBP of each beat. The slopes are determined by the linear regression of the filtered BP beat values as a function of time in seconds.

In terms of de novo delta RRI magnitude increase in AF individuals, FIG. 13 shows a distinct increase in the magnitude of the beat-to-beat RRI changes in the last read aloud interval, labeled RAI, of the read aloud PADL session. As with detecting most or all de novo deviations, in an embodiment, the detection of a de novo increase in RRI magnitude is performed on a progressive cyclic basis. In general, the detection process may determine if the set of data from the current read aloud PADL cycle is statistically improbable given the ensemble data from all of the previous cycles. If it is significantly improbable, then this represents a discontinuity in probability, and the de novo emergence of a new entity. In FIG. 13, the first 4 cycles of read aloud PADL are of low challenge level and are relatively short in duration, yielding only a few new data points per cycle. Thus for detection purposes, in this embodiment, the first 4 cycles are initially pooled together to provide a larger first ensemble data set of previous data. Thus, de novo entity detection begins at cycle 5 and progresses to the end of the measured read aloud PADL response. In this case, the measured data is the absolute value of the delta RRI from the previous beat. All non-PVC beats, including those from inspiration and expiration are used for detection. It is to be appreciated that in other embodiments, a different number of initial read aloud cycles may be pooled together. This technique for detecting a deviation is generally only usable with AF individuals, meaning it typically is not usable with non-AF individuals.

In an embodiment, the method of assessing health risk or performance of an individual according to the present disclosure involves generating CV risk score information. A CV risk score may be generated based on the comparing the CV response of the individual to a PADL to reference CV response information and/or determining if the CV response manifests one or more deviations. A low score may indicate a low CV risk and thus a low health risk for the individual, whereas a high score may indicate a high CV risk and thus a high health risk for the individual.

The CV risk score may be based on or calculated using a point system where a predetermined value, such as a number of points, is assigned when a given deviation is detected in the CV response of the individual. When multiple deviations are detected, the points associated with each deviation may be added up to generate an aggregate CV risk score. As an example, CV risk score=x points (deviation A)+y points (deviation C)+z points (deviation F), and where deviations B, D, E, and G were not detected. In an embodiment, the number of points assigned for a given deviation may be weighted based on some factor such as the severity of the deviation. Further, the number of points associated with the different deviations may have different sensitivity, specificity, and/or predictive accuracy dependent on the population being tested. Thus the exact weighting of the points associated with the deviations may be changed or optimized for individual population characteristics. For example, for one population being tested, a SBP alternans deviation may be assigned 3 points, whereas for a different population the deviation may be assigned 4 points.

Some example point assignments for various deviations are now provided.

The detection of a de novo SBP alternans detected during read aloud PADL, independent of RRI alternans, may be assigned 3 points. For example, in an embodiment, SBP alternans having magnitudes of >3 mm Hg or > than 5% of the mean pulse pressure, or a SBP alternans diff % of >15%, is associated with increased risk of re-hospitalization (FIG. 6). In an embodiment, the number of points assigned may correspond to a severity of the deviation. The definition of SBP alternans diff % is the percentage of the sum of the absolute differences of SBP for all alternans beats divided by the sum of the absolute differences of SBP for all beats. (3 points). These threshold values may vary and may depend on the population tested.

The detection of de novo read aloud PADL emergence of PVCs or APCs or more complex arrhythmias may be assigned 2 points. For example in FIG. 7, there is a high frequency of premature beats in the second half of the read aloud PADL session compared to the first half of the session.

The detection of lack of inspiratory relative tachycardia and loss of expiratory bradycardia that together create a bradycardic capacity failure, and may be assigned 1 point. For example, the top graph in FIG. 10 showing RRI shows the diminishing capacity to produce

RRI modulation in response to the read aloud PADL.

The detection of sharp inspiratory SBP drop within a prescribed number of inspiratory beats may be assigned 1 point. For example, a sharp blood pressure drop during inspiration is shown in FIG. 9.

The detection of progressive relative tachycardia during read aloud may be assigned 1 point. For example, the top graph in FIG. 10 showing RRI shows progressive tachycardia during read aloud cycles.

The detection of a failure in DBP to modulate in response to a read aloud PADL session may be assigned 1 point, and a failure in PP to modulate may be assigned 1 point. Specifically, a significant difference is generally expected between the de-trended inspiration BP values (low-pass filtered at a low frequency such as 0.1 Hz) and the de-trended expiratory BP values (a total 2 points). This concept for PP is shown in FIG. 8. For instance, PP, unlike DBP and SBP, in FIG. 8 is not modulating in response to the individual breathe-in/reading aloud cycles.

The detection of progressive hypertension in either DBP or PP, or any de novo read aloud PADL induced changes in shape, may be assigned 1 point. For example FIG. 11 shows a progressive increase in DBP during the PADL session.

The detection of an unsynchronized oscillatory very low frequency changes in any blood pressure (not phase coupled to the read aloud script) with wavelength of 30-60 seconds may be assigned 1 point.

The above point assignments for various deviations are examples only and are not limiting. Again, the point values assigned may be different depending on one or more factors such as the population being tested or the weighted effect that the presence of the deviation has on the CV health of the individual.

The exact details of the implementation of the read aloud PADL protocols may vary depending on the user population and the goals of the testing. For example, in a designated healthy elite athlete, there may be no limits to the number of read aloud cycles that are used. In individuals with near end stage heart failure, it may be prudent to limit the number of read aloud cycles or there may be a need for on the fly analysis that would terminate the read aloud protocol to an incipient excess physiologic challenge. There may be different preconditions that may be used to increase or decrease predictive capacity. For example, during transients such as viral infections or a myriad other medical situations (joint probability analysis).

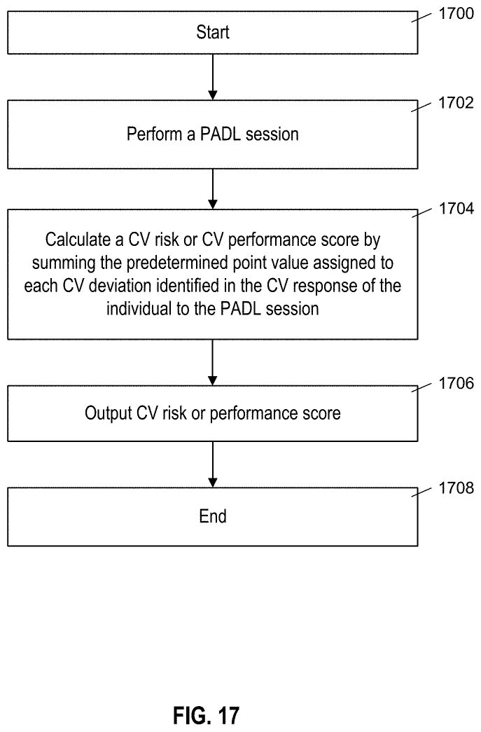

FIG. 17 is a process flow chart of an example method of a read aloud PADL according to the present disclosure. The process begins at block 1700 and proceeds to block 1702 where a PADL session is performed on an individual. The process proceeds to block 1704 where a CV risk or CV performance score is calculated by summing the predetermined point value assigned to each CV deviation identified in the CV response of the individual in the PADL session. The process proceeds to block 1706 where the score is outputted, for example from a computing device that calculated the score. The process proceeds to block 1708 and ends.

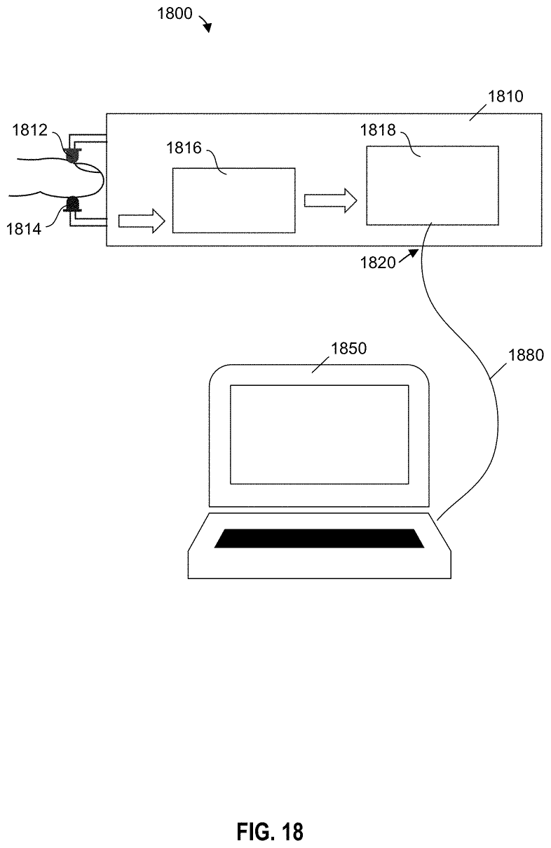

FIG. 18 is a block diagram of an example system 1800 comprising a physiological sensor device 1810 in communication with a computing device 1850 that may be used to perform a read aloud PADL session. Sensor device 1810 may be a light emitting diode (LED)/photo-transistor sensor for measuring one or more physiological parameters of an individual, including CV parameters such as blood pressure and/or pulse. Sensor device 1810 may be used on an extremity of the body, such as a finger, and may comprise an infrared LED 1812 paired with a photo-transistor 1814. LED 1812 shines light through the extremity where it is sensed by photo-transistor 1814 to generate measurement signals, such as BP proxy values. Measurement signals, for example signals from photo-transistor 1814, may be processed before they are outputted from sensor device 1810. For example, signals from photo-transistor 1814 may be band-pass filtered at filter 1816. In an embodiment, the filter is configured with a frequency pass band of approximately 0.03 Hz to 50 Hz, and a gain of approximately 100. Further, measurement signals may be converted into digital by an analog-to-digital converter (ADC) 1818. In an embodiment, ADC 1818 may be a 12-bit ADC and may have a sampling rate of 100 samples per second. ADC 1818 may be implemented using an embedded microcontroller. The digitally converted signal may be outputted from sensor device 1810 and communicated to computing device 1850 over communication link 1880, such as a Bluetooth Low Energy link.

Computing device 1850 may receive the signal from sensor device 1810 and may perform further processing. The processing may include any of the measuring, comparing, determining or other processing relating to a PADL session. For example, the processing may include the comparing at least one of the CV responses of the individual to predefined reference CV response information, and/or determining, based on the comparing, if at least one of the CV responses represent a CV response deviation relative to the reference CV response information.

In other embodiments and as would be understood by a person skilled in the art, system 1800 may differ from the one described above. For instance, physiological sensor device 1810 may be a different sensor or set of sensors, or may be configured differently. For example, one or both of filter 1816 and ADC 1818 may be configured differently than described above.

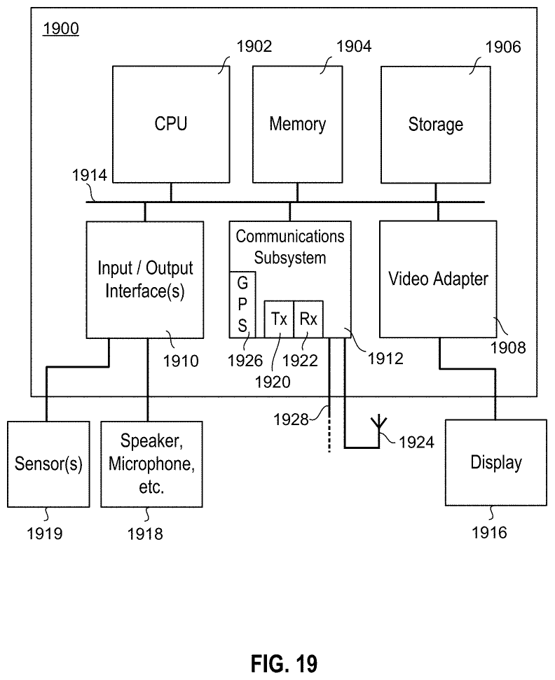

Aspects according to the present disclosure may be implemented on any suitable apparatus or apparatuses. In at least some embodiments, the apparatus includes one or more computing devices and/or computer related components. FIG. 19 is a block diagram of an example electronic device or system 1900. The electronic device may be any suitable type of device, including but not limited to a mobile device, a smartphone, a tablet, a notebook computer, a desktop computer, a server, and a mainframe.

The teachings of the present disclosure may be implemented at or performed by any network element or combination of network elements. A network element may be a network side electronic device, such as a server, or a user side electronic device, such as mobile device or other personal electronic device. These network side and user side devices are only examples and are not intended to be limiting.

The electronic device or system 1900 may include one or more of a central processing unit (CPU) 1902, memory 1904, a mass storage device 1906, a video adapter 1908, an input/output (I/O) interface 1910, and a communications subsystem 1912. One or more of the components or subsystems of electronic device 1900 may be interconnected by way of one or more buses 1914 or in any other suitable manner.

The bus 1914 may be one or more of any type of several bus architectures including a memory bus or memory controller, a video bus, peripheral bus, or the like. The CPU 1902 may comprise any type of electronic data processor. CPU 1902 may be configured to perform operations according to the present disclosure, for example including but not limited to comparing a CV response to predefined reference CV response information, and/or determining if a CV responses represents a CV response deviation relative to the reference CV response information.

The memory 1904 may comprise any type of system memory such as dynamic random access memory (DRAM), static random access memory (SRAM), synchronous DRAM (SDRAM), read-only memory (ROM), a combination thereof, or the like. In an embodiment, the memory may include ROM for use at boot-up, and DRAM for program and data storage for use while executing programs.

The mass storage device 1906 may comprise any type of storage device configured to store data, programs, and other information and to make the data, programs, and other information accessible via the bus 1914. The mass storage device may comprise, for example, one or more of a solid state drive, hard disk drive, a magnetic disk drive, an optical disk drive, or the like. In some embodiments, data, programs, or other information may be stored remotely, for example in the “cloud”. Electronic device 1900 may send or receive information to the remote storage in any suitable way, including via communications subsystem 1912 over a network or other data connection.

The video adapter 1908 and the I/O interface 1910 may provide interfaces to couple external input and output devices to the electronic device. As illustrated, examples of input and output devices include a display 1916, such as an electronic display, coupled to the video adapter 1908 and the LED, speaker, or microphone 1918 coupled to the I/O interface 1910. Display 1916 may be used to display information during a read aloud PADL session, such as the sequence of information sets to be read aloud and intervening “breathe-in” messages. In addition, one or more sensors 1919 may be coupled to I/O interface 1910. The one or more sensors 1919 may include any suitable type of sensor, including but not limited to one or more physiological sensors for measuring CV responses and/or other CV parameters of an individual. For example, one or more sensors 1919 may be one of sensors 110 in FIG. 1 or sensor 1810 in FIG. 18, respectively. It is to be appreciated, however, that these peripherals and other devices are examples only. Other devices may be coupled or connected to the electronic device in addition to or in place of those shown and described. Furthermore, additional or fewer interfaces may be utilized. For example, one or more serial interfaces such as Universal Serial Bus (USB) (not shown) may be provided.

A communications subsystem 1912 may be provided for one or both of transmitting and receiving signals. Communications subsystems may include any component or collection of components for enabling communications over one or more wired and wireless interfaces. These interfaces may include but are not limited to USB, Ethernet, high-definition multimedia interface (HDMI), Firewire (e.g. IEEE 1394), Thunderbolt™, WFi™ (e.g. IEEE 802.11), WiMAX (e.g. IEEE 802.16), Bluetooth™, or Near-field communications (NFC), as well as GPRS, UMTS, LTE, LTE-A, dedicated short range communication (DSRC), and IEEE 802.1 1. Furthermore, any of the communication links over which device 1900 or any of its components communicate may be either wired and/or wireless links. This includes, for example, communication links with display 1916, speaker, etc. 1918, and/or sensor(s) 1919.

Communication subsystem 1912 may include one or more ports or other hardware 1928 for one or more wired connections. In addition, communication subsystem 1912 may include one or more of transmitters 1920, receivers 1922, and antenna elements 1924. In at least some embodiments, the electronic device may have geographic positioning functionality, for example to determine a geographical position of the electronic device or for receiving timing signals for time synchronization of the device with other systems. In at least some embodiments, the electronic device may be capable of receiving Global Positioning System (GPS) signals. Therefore in at least one embodiment, as shown in FIG. 19, the electronic device may comprise a GPS radio or receiver 1926. However, other embodiments may comprise and use other subsystems or components for, for example, determining the geographical position of the electronic device or for receiving timing signals for time synchronization. In some embodiments, the electronic device may be configured to determine a geographic location using WiFi.

The electronic device 1900 of FIG. 19 is merely an example and is not meant to be limiting. Various embodiments may utilize some or all of the components shown or described. Some embodiments may use other components not shown or described but known to persons skilled in the art. Furthermore, a device may contain multiple instances of a component, such as multiple electronic device, processors, memories, transmitters, receivers, etc. The electronic device may comprise one or more input/output devices, such as a speaker, microphone, mouse, touchscreen, keypad, keyboard, display, and the like. Various other options and configurations are contemplated.

In addition, the present disclosure contemplates devices, apparatuses, methods and systems according with the teachings of the above description and appended drawings.

Through the descriptions of the preceding embodiments, the teachings of the present disclosure may be implemented by using hardware only or by using a combination of software and hardware. Software or other computer executable instructions for implementing one or more embodiments, or one or more portions or aspects thereof, may be stored on any suitable computer readable storage medium. The computer readable storage medium may be or comprise a non-transitory medium such as optical (e.g., CD, DVD, Blu-Ray, etc.), magnetic, hard disk, volatile or non-volatile, solid state, or any other type of storage medium known in the art.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether embodiments or portions thereof, which are described herein, are implemented as a software routine, hardware circuit, firmware, or any combination thereof.

Embodiments of the disclosure may be represented as a computer program product stored in a machine-readable medium (also referred to as a computer-readable medium, a processor-readable medium, or a computer usable medium having a computer-readable program code embodied therein). The machine-readable medium may be any suitable tangible, non-transitory medium, including magnetic, optical, or electrical storage medium including a diskette, compact disk read only memory (CD-ROM), memory device (volatile or non-volatile), or similar storage mechanism. The machine-readable medium may contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations may also be stored on the machine-readable medium or media. The instructions stored on the machine-readable medium may be executed by a processor or other suitable processing device, and may interface with circuitry to perform the described tasks.

The structure, features, accessories, and alternatives of specific embodiments described herein and shown in the Figures are intended to apply generally to all of the teachings of the present disclosure, including to all of the embodiments described and illustrated herein, insofar as they are compatible. In other words, the structure, features, accessories, and alternatives of a specific embodiment are not intended to be limited to only that specific embodiment unless so indicated.

In addition, the steps and the ordering of the steps of methods and processes described herein are not meant to be limiting. Methods comprising different steps, different number of steps, and/or different ordering of steps are also contemplated.

For simplicity and clarity of illustration, reference numerals may have been repeated among the figures to indicate corresponding or analogous elements. Numerous details have been set forth to provide an understanding of the embodiments described herein. The embodiments may be practiced without these details. In other instances, well-known methods, procedures, and components have not been described in detail to avoid obscuring the embodiments described.

The above-described embodiments are intended to be examples only. Alterations, modifications and variations can be effected to the particular embodiments by those of skill in the art without departing from the scope, which is defined solely by the claims appended hereto.

Some abbreviations used herein:

CV: cardiovascular
BP: blood pressure
SBP: systolic blood pressure
DBP: diastolic blood pressure
PP: pulse pressure
RRI: R-R interval
PVC: premature ventricular contraction
APC: atrial premature contraction
AF: atrial fibrillation
PADL: programmed activities of daily living

What is claimed is:

1. A method of assessing health performance of a human, the method comprising:
displaying sets of information sequentially in time to be read aloud by the human, wherein each set of information is configured to serve as a physiological micro challenge to the human when read aloud, and wherein on average the size of the information in each set of information increases over time thereby increasing the intensity of the micro challenges;
measuring a sequence of cardiovascular (CV) responses of the human to each of the physiological micro challenges during a session, each of the CV responses associated in time with the reading aloud of one of the sets of information;
comparing at least one of the CV responses to predefined reference CV response information; and
determining, based on the comparing, if at least one of the CV responses represents a CV response deviation relative to the reference CV response information.

2. The method of claim 1,
wherein the determining involves determining if one or more of the measured CV responses indicates a CV response deviation in the form of a heart beat scale CV abnormality that emerged during the session of measured CV responses, wherein the CV abnormality is abnormal relative to a reference heart beat scale CV response in the reference CV response information.

3. The method of claim 2, wherein the heart beat scale CV abnormality is at least one of a blood pressure alternans or premature ventricular contractions (PVC).

4. The method of claim 1,
wherein the measuring a CV response to a given physiological micro challenge involves measuring the CV response spanning a read aloud interval, where a read aloud interval is the time period during which the set of information corresponding to the given micro challenge is read aloud, and
wherein the determining involves determining if there is a CV response deviation in the form of an abnormality in the way in which the read aloud interval CV responses of the human change during the session of measured CV responses, where the change is abnormal relative to reference read aloud interval CV response change information.

5. The method of claim 4, wherein the abnormality in the way in which the read aloud interval CV responses of the human change is in the form of at least one of a blood pressure (BP) change and a heart rate (HR) change.

6. The method of claim 1, wherein the determining involves determining if there is a CV response deviation in the form of an average incremental or decremental change in a CV characteristic during the session of measured CV responses that is abnormal relative to reference change information for the CV characteristic.

7. The method of claim 6, wherein the average incremental or decremental change in a CV characteristic includes at least one of a progressive relative tachycardia and a blood pressure progressive hypertension.

8. The method of claim 1, further comprising measuring CV responses of the human during breathe-in intervals that interspace read aloud intervals during which the sets of information are read aloud.

9. The method of claim 1, wherein the session terminates upon a first occurrence of a failure of one of the displayed sets of information to be read aloud.

10. The method of claim 9, wherein the termination occurs due to self-termination by the human in response to the intensity of the micro challenges reaching an intolerable level to the human.

11. The method of claim 1, wherein the measuring a sequence of cardiovascular (CV) responses involves measuring a blood pressure of the human.

12. The method of claim 1, further comprising generating an output indicating the result of the determination.

13. A system comprising:
a computer processor;
a computer memory in communication with the processor;
a display in communication with the processor;
a physiological sensor in communication with the processor,
wherein the processor, display and sensor are configured to:
display sets of information sequentially in time to be read aloud by the human, wherein each set of information is configured to serve as a physiological micro challenge to the human when read aloud, and wherein on average the size of the information in each set of information increases over time thereby increasing the intensity of the micro challenges;
measure a sequence of cardiovascular (CV) responses of the human to each of the physiological micro challenges during a session, each of the CV responses associated in time with the reading aloud of one of the sets of information;
compare at least one of the CV responses to predefined reference CV response information; and
determine, based on the comparing, if at least one of the CV responses represent a CV response deviation relative to the reference CV response information.

14. The system of claim 13, wherein the determining involves determining if one or more of the measured CV responses indicates a CV response deviation in the form of a heart beat scale CV abnormality that emerged during the session of measured CV responses, wherein the CV abnormality is abnormal relative to a reference heart beat scale CV response in the reference CV response information.

15. The system of claim 14, wherein the heart beat scale CV abnormality is at least one of a blood pressure alternans or premature ventricular contractions (PVC).

16. The system of claim 13,
wherein the measuring a CV response to a given physiological micro challenge involves measuring the CV response spanning a read aloud interval, where a read aloud interval is the time period during which the set of information corresponding to the given micro challenge is read aloud, and
wherein the determining involves determining if there is a CV response deviation in the form of an abnormality in the way in which the read aloud interval CV responses of the human change during the session of measured CV responses, where the change is abnormal relative to reference read aloud interval CV response change information.

17. The system of claim 13,
wherein the determining involves determining if there is a CV response deviation in the form of an average incremental or decremental change in a CV characteristic during the session of measured CV responses that is abnormal relative to reference change information for the CV characteristic.

18. The system of claim 17,
wherein the average incremental or decremental change in a CV characteristic includes at least one of a progressive relative tachycardia and a blood pressure progressive hypertension.

19. The system of claim 13, further configured to measure CV responses of the human during breathe-in intervals that interspace read aloud intervals during which the sets of information are read aloud.

20. A non-transitory computer-readable medium comprising computer readable instructions which, when executed by one or more processors, cause the performance of:
displaying sets of information sequentially in time to be read aloud by the human, wherein each set of information is configured to serve as a physiological micro challenge to the human when read aloud, and wherein on average the size of the information in each set of information increases over time thereby increasing the intensity of the micro challenges;
measuring a sequence of cardiovascular (CV) responses of the human to each of the physiological micro challenges during a session, each of the CV responses associated in time with the reading aloud of one of the sets of information;
comparing at least one of the CV responses to predefined reference CV response information; and
determining, based on the comparing, if at least one of the CV responses represents a CV response deviation relative to the reference CV response information.
